(19) United States
(12) Patent Application Publication
(10) Pub. No.: US 2024/0293549 A1
(43) Pub. Date: Sep. 5, 2024

(54) Conjugates And Conjugating Reagents

(71) Applicant: POLYTHERICS LIMITED, Cambridge (GB)

(72) Inventors: Antony Godwin, Cambridge (GB); Mark Frigerio, Cambridge (GB)

(73) Assignee: POLYTHERICS LIMITED, Cambridge (GB)

(21) Appl. No.: 18/609,210

(22) Filed: Mar. 19, 2024

Related U.S. Application Data
(63) Continuation of application No. 16/807,298, filed on Mar. 3, 2020.
Application No. 16/807,298 is a continuation of application No. 15/517,969, filed on Apr. 10, 2017, now abandoned.

(30) Foreign Application Priority Data
Oct. 24, 2014 (GB) 1418984.9
Oct. 24, 2014 (GB) 1418986.4
Oct. 24, 2014 (GB) 1418989.8

(51) Int. Cl. A61K 47/10; A61K 31/33; A61K 47/60; A61K 47/68

(52) U.S. Cl. CPC A61K 47/10; A61K 31/33; A61K 47/60; A61K 47/68; A61K 47/6851; A61K 47/6889

(57) ABSTRACT

The invention relates to a conjugate of a protein or peptide with a therapeutic, diagnostic or labelling agent, said conjugate containing a protein or peptide bonding portion and a polyethylene glycol portion; in which said protein or peptide bonding portion has the general formula:

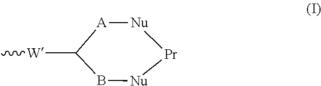

in which Pr represents said protein or peptide, each Nu represents a nucleophile present in or attached to the protein or peptide, each of A and B independently represents a C₁₋₄alkylene or alkenylene chain, and W′ represents an electron withdrawing group or a group obtained by reduction of an electron withdrawing group; and in which said polyethylene glycol portion is or includes a pendant polyethylene glycol chain which has a terminal end group of formula —CH₂CH₂OR in which R represents a hydrogen atom, an alkyl group, or an optionally substituted aryl group. Also claimed are a method for making such a conjugate, and novel reagents useful in that method.

### FIELD OF INVENTION

This invention relates to novel conjugates and novel conjugating reagents.

### BACKGROUND OF THE INVENTION

Much research has been devoted in recent years to the conjugation of a wide variety of payloads, for example therapeutic, diagnostic and labelling agents, to peptides and proteins for a wide range of applications. The protein or peptide itself may have therapeutic properties, and/or it may be a binding protein.

Peptides and proteins have potential use as therapeutic agents, and conjugation is one way of improving their properties. For example, water soluble, synthetic polymers, particularly polyalkylene glycols, are widely used to conjugate therapeutically active peptides or proteins. These therapeutic conjugates have been shown to alter pharmacokinetics favourably by prolonging circulation time and decreasing clearance rates, decreasing systemic toxicity, and in several cases, displaying increased clinical efficacy. The process of covalently conjugating polyethylene glycol, PEG, to proteins is commonly known as “PEGylation”. The PEG chain may carry a payload, for example a therapeutic, diagnostic or labelling agent.

Binding proteins, particularly antibodies or antibody fragments, are frequently conjugated. The specificity of binding proteins for specific markers on the surface of target cells and molecules has led to their extensive use either as therapeutic or diagnostic agents in their own right or as carriers for payloads which may include therapeutic, diagnostic or labelling agents. Such proteins conjugated to labels and reporter groups such as fluorophores, radioisotopes and enzymes find use in labelling and imaging applications, while conjugation to drugs such as cytotoxic agents and chemotherapy drugs to produce antibody-drug conjugates (ADCs) allows targeted delivery of such agents to specific tissues or structures, for example particular cell types or growth factors, minimising the impact on normal, healthy tissue and significantly reducing the side effects associated with chemotherapy treatments. Such conjugates have extensive potential therapeutic applications in several disease areas, particularly in cancer. Conjugates containing binding proteins frequently contain PEG.

Many methods of conjugating proteins and peptides have been reported in the literature. Probably the most commonly used process involves the use of conjugating reagents based on maleimides. Such reagents are described in many publications, for example WO 2004/060965. An alternative approach which leads to more homogeneous products is described by Liberatore et al, Bioconj. Chem 1990, 1, 36-50, and del Rosario et al, Bioconj. Chem. 1990, 1, 51-59, which describe the use of reagents which may be used to cross-link across the disulfide bonds in proteins, including antibodies. WO 2005/007197 describes a process for the conjugation of polymers to proteins, using novel conjugating reagents having the ability to conjugate with both sulfur atoms derived from a disulfide bond in a protein to give novel thioether conjugates, while WO 2009/047500 describes the use of the same conjugating reagents to bond to polyhistidine tags attached to the protein. WO 2010/000393 describes reagents capable of forming a single carbon bridge across the disulfide bond in a protein. Other documents relating to the conjugation of proteins include WO 2014/064423, WO 2013/190292, WO 2013/190272 and EP 2260873.

WO 2014/064424 describes specific ADCs in which the drug is a maytansine and the antibody is bonded by cross-linking across a disulfide bond. WO 2014/064423 describes specific ADCs in which the drug is an auristatin and the antibody is bonded by cross-linking across a disulfide bond. The linkers illustrated in the Examples of these documents contain a PEG portion in which one end of the PEG chain is attached via a further portion of the linker to the drug, while the other end of the PEG chain is attached via a further portion of the linker to the antibody. This is a common structural pattern for ADCs.

Over recent years, the importance of the linker which links a payload to the protein or peptide in a conjugate, has become apparent. Often, the key decision to be taken whether it is desired to have a cleavable linker, i.e. a linker which, on administration of the conjugate, degrades to release the free payload, or a non-cleavable linker. Another key decision is whether or not to include PEG in the linker. Subject to these considerations, in principle, any linker may be used. In practice, however, changes in structure of the linker may lead to differences in the properties either of the conjugating reagent or of the resulting conjugate.

One problem frequently found is that conjugates may be less storage stable than desired. This is particularly true when a cleavable linker is used, when it is desired that the conjugate should have a long shelf-life before administration but should then rapidly cleave on application, but it can be true for any linker. There is a need to increase the storage stability of conjugates. In addition, improved stability in vivo is desirable, as this can lead to increased biological activity. We have now found that for a particular class of conjugate, the use of PEG-containing linkers of a particular structure gives increased storage stability. Further, and very surprisingly, the conjugates have increased biological activity.

### SUMMARY OF THE INVENTION

The invention provides a conjugate of a protein or peptide with a therapeutic, diagnostic or labelling agent, said conjugate containing a protein or peptide bonding portion and a polyethylene glycol portion; in which said protein or peptide bonding portion has the general formula:

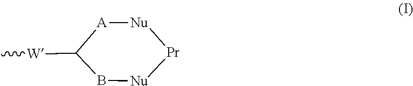

in which Pr represents said protein or peptide, each Nu represents a nucleophile present in or attached to the protein or peptide, each of A and B independently represents a C₁₋₄alkylene or alkenylene chain, and W′ represents an electron withdrawing group or a group obtained by reduction of an electron withdrawing group; and in which said polyethylene glycol portion is or includes a pendant polyethylene glycol chain which has a terminal end group of formula —CH₂CH₂OR in which R represents a hydrogen atom, an alkyl group, for example a C₁₋₄alkyl group, especially a methyl group, or an optionally substituted aryl group, especially a phenyl group, especially an unsubstituted phenyl group.

The invention also provides a conjugating reagent capable of reacting with a protein or peptide, and including a therapeutic, diagnostic or labelling agent and a polyethylene glycol portion; said conjugating reagent including a group of the formula:

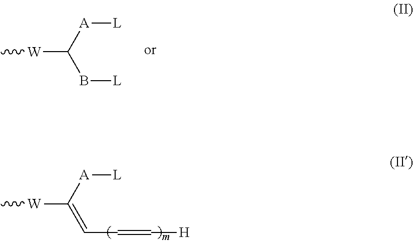

in which W represents an electron withdrawing group, A and B have the meanings given above, m is 0 to 4, and each L independently represents a leaving group; and in which said polyethylene glycol portion is or includes a pendant polyethylene glycol chain which has a terminal end group of formula —CH₂CH₂OR in which R represents a hydrogen atom, an alkyl group, for example a C₁₋₄alkyl group, especially a methyl group, or an optionally substituted aryl group, especially a phenyl group, especially an unsubstituted phenyl group.

The invention also provides a process for the preparation of a conjugate according to the invention, which comprises reacting a protein or peptide with a conjugating reagent according to the invention.

### DETAILED DESCRIPTION OF THE INVENTION

The conjugate of the invention may be represented schematically by the formula:

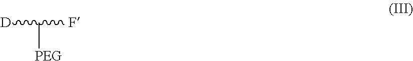

in which D represents the therapeutic, diagnostic or labelling agent, F′ represents the group of formula I, and PEG represents the pendant polyethylene glycol chain having a terminal end group of formula —CH₂CH₂OR.

The reagent of the invention may be represented schematically by the formula:

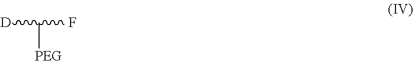

in which D represents the therapeutic, diagnostic or labelling agent, F represents the group of formula II or II′, and PEG represents a pendant polyethylene glycol chain having a terminal end group of formula —CH₂CH₂OR. The functional grouping F is capable of reacting with two nucleophiles present in a protein or peptide as explained below.

### The Polyethylene Glycol Portion

A polyethylene glycol (PEG) portion of the conjugates and reagents of the invention is or includes a pendant PEG chain which has a terminal end group of formula —CH₂CH₂OR in which R represents a hydrogen atom, an alkyl group, for example a C₁₋₄alkyl group, especially a methyl group, or an optionally substituted aryl group, especially a phenyl group, especially an unsubstituted phenyl group. Preferably R is a methyl group or a hydrogen atom.

The PEG portion may include a single pendant PEG chain as defined above, or it may include two or more, for example two or three, pendant PEG chains.

The overall size of the PEG portion will of course depend on the intended application. For some applications, high molecular weight PEGs may be used, for example the number average molecular weight may be up to around 75,000, for example up to 50,000, 40,000 or 30,000 g/mole. For example, the number average molecular weight may be in the range of from 500 g/mole to around 75,000. However, smaller PEG portions may be preferred for some applications.

In one preferred embodiment, all of the PEG in the PEG portion is present in one or more pendant PEG chains. In another embodiment, PEG may also be present in the backbone of the molecule, and this is discussed in more detail below.

As with the PEG portion, the size of the pendant PEG chain or chains will depend on the intended application. For some applications, high molecular weight pendant PEG chains may be used, for example the number average molecular weight may be up to around 75,000, for example up to 50,000, 40,000 or 30,000 g/mole. For example, the number average molecular weight may be in the range of from 500 g/mole to around 75,000. However, for many applications, smaller pendant PEG chains may be used. For example said PEG chain may have a molecular weight up to 3,000 g/mole. However, very small oligomers, consisting of discrete PEG chains with, for example, as few as 2 repeat units, for example from 2 to 50 repeat units, are useful for some applications, and are present as a pendant PEG chain in one preferred embodiment of the invention. A pendant PEG chain may be straight-chain or branched. PEG chains, for example straight-chain or branched chains with 12, 20, 24, 36, 40 or 48 repeat units may for example be used.

### The Payload

The conjugates and reagents of the invention carry a payload which is a therapeutic, diagnostic or labelling agent. A single molecule of a therapeutic, diagnostic or labelling agent may be present, or two or more molecules may be present. The inclusion of one or more drug molecules, for example a cytotoxic agent or a toxin, is preferred. Auristatins and maytansinoids are typical cytotoxic drugs. It is often preferred that drug conjugates, particularly antibody drug conjugates, should contain multiple copies of the drug. Labelling agents (which should be understood to include imaging agents) may for example include a radionuclide, a fluorescent agent (for example an amine derivatised fluorescent probe such as 5-dimethylaminonaphthalene-1-(N-(2-aminoethyl))sulfonamide-dansyl ethylenediamine, Oregon Green® 488 cadaverine (catalogue number O-10465, Molecular Probes), dansyl cadaverine, N-(2-aminoethyl)-4-amino-3,6-disulfo-1,8-naphthalimide, dipotassium salt (lucifer yellow ethylenediamine), or rhodamine B ethylenediamine (catalogue number L 2424, Molecular Probes), or a thiol derivatised fluorescent probe for example BODIPY® FL L-cystine (catalogue number B-20340, Molecular Probes). Biotin may also be used.

Our copending application GB 1418984 provides a conjugate comprising a protein, peptide and/or polymer attached to a maytansine-containing payload via a linker, a conjugating reagent useful in forming such conjugates and a maytansine-containing compound for use as payload. The maytansine-containing payloads and compounds consist of at least two maytansine moieties linked to each other through a non-degradable bridging group. These maytansines may be used as payloads in the present invention, and the reagents and conjugates form one aspect of the present invention. Our copending application discloses the following:
“The present invention provides in a first aspect a maytansine-containing compound, in which at least two maytansine moieties (D) are linked to each other through a bridging group (Bd). The bridging group (Bd) is non-degradable under physiological conditions. Advantageously, the bridging group (Bd) has at least 3 chain carbon atoms and optionally contains poly(ethylene glycol) spacers in addition to the 3 chain carbon atoms. Advantageously, no two heteroatoms are adjacent to one another in the bridging group. Advantageously, the bridging group does not include the moiety: —C(O)—CH(NR¹X)—(CH₂)_{b}—C(O)—, where bis 1, 2 or 3, R¹ is selected from hydrogen and C₁ to C₆ alkyl, and X is any group. The maytansine-containing compound of the first aspect, in which two maytansine moieties are present, may be represented by the following formula (I):

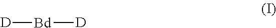

In a second aspect, the invention provides a conjugating reagent, which contains a functional group capable of reaction with a peptide or protein and/or a functional group capable of reacting with a polymer, the payload being attached to the functional group(s) via one or more linkers, characterised in that the conjugation reagent comprises a maytansine-containing payload consisting of at least two maytansine moieties linked to each other through a non-degradable bridging group, with the proviso that when the conjugating reagent comprises a functional group capable of reaction with at a peptide or protein, the linker attaching the payload to the functional group capable of reaction with at a peptide or protein is degradable. When the conjugating reagent contains a functional group capable of reaction with at least one nucleophile present in a peptide or protein, the functional group including at least one leaving group which is lost on reaction with said nucleophile, the maytansine-containing payload consisting of at least two maytansine moieties linked to each other through a non-degradable bridging group is advantageously attached to the functional group capable of reaction with at least one nucleophile present in a peptide or protein via a degradable linker. The conjugating reagent optionally contains a functional group capable of reaction with at least one nucleophile present in a peptide or protein, the functional group advantageously including at least one leaving group which is lost on reaction with said nucleophile, characterised in that the conjugation reagent comprises a maytansine-containing payload consisting of at least two maytansine moieties, especially two maytansine moieties, linked to each other through a non-degradable bridging group, and in that the payload is attached to the functional group capable of reaction with at least one nucleophile present in a peptide or protein via a linker, especially a degradable linker. The linker is suitable for linking the bridging group to a protein or peptide capable of binding to a partner or target. Preferably, the bridging group (Bd) of the maytansine-containing payload (D₂Bd) is connected to a degradable linker (Lk^{d}) that includes a degradable group which breaks under physiological conditions. The degradable group may, for example, be sensitive to hydrolytic conditions, especially acidic conditions; be susceptible to degradation under reducing conditions; or be susceptible to enzymatic degradation.
The maytansine-containing conjugating reagent of the second aspect, in which two maytansine moieties are present, may be represented by the following formula (II):

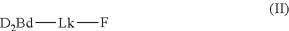

in which D₂Bd represents a maytansine-containing payload consisting of two maytansine moieties linked to each other through a non-degradable bridging group, Lk is a linker, especially a degradable linker (Lk^{d}), and F represents a functional group capable of reaction with a peptide or protein and/or a functional group capable of reacting with a polymer.
The present invention further provides a process for the conjugation of a peptide, protein and/or a polymer, which comprises reacting said peptide, protein and/or a polymer with a conjugating reagent of the second aspect of the invention. When the conjugating reagent is reacted with a peptide or polymer, said conjugating reagent is advantageously capable of reaction with at least one nucleophile present in said peptide or protein, said reagent advantageously containing at least one leaving group which is lost on reaction with said nucleophile.
The invention also provides in a third aspect a conjugate comprising a protein, peptide and/or polymer attached to a maytansine-containing payload via a linker, characterised in that the maytansine-containing payload consists of at least two maytansine moieties linked to each other through a non-degradable bridging group. When the conjugate comprises a protein or peptide, the linker attaching the payload to the protein or peptide is advantageously degradable. The conjugate of the third aspect of the invention may, for example may, for example, comprise a maytansine-containing drug moiety (D₂Bd) linked via a linker (Lk), especially a degradable linker (Lk^{d}), to a protein or peptide (Ab) capable of binding to a partner or target, wherein the maytansine-containing drug moiety (D₂Bd) comprises at least two maytansine moieties (D) linked to each other through a non-degradable bridging group (Bd). A maytansine-containing conjugate of the third aspect of the invention, in which two maytansine moieties (D) are present, may be represented by the following formula (III):

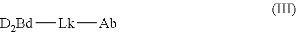

The linker (Lk) is advantageously a degradable linker (Lk^{d}) that includes a degradable group which cleaves under physiological conditions separating the maytansine-containing drug moiety (D₂Bd) comprising at least two maytansine moieties (D) linked to each other through a bridging group (Bd) from the protein or peptide (Ab) capable of binding to a partner or target. The degradable group may, for example, be sensitive to hydrolytic conditions, especially acidic conditions; be susceptible to degradation under reducing conditions; or be susceptible to enzymatic degradation. The non-degradable bridging group (Bd) contains no groups that are susceptible to cleavage under the same conditions as those under which the degradable group in the degradable linker cleaves.
The maytansine-containing compound of the first aspect of the invention, may, for example, comprise or consist of at least two maytansine moieties (D), especially two maytansine moieties, linked to each other through a bridging group (Bd) having at least 3 chain carbon atoms, especially at least 7 chain carbon atoms, and optional poly(ethylene glycol) units in addition to the chain carbon atoms, with the proviso that no two heteroatoms are adjacent to one another in the bridging group and with the proviso that the bridging group does not include the moiety: —C(O)—CH(NR¹X)—(CH₂)_{b}—C(O)—, where b is 1, 2 or 3, R¹ is selected from hydrogen and C₁ to C₆ alkyl, and X is any group. Optionally, the bridging group incorporates from 0 to 8 carbonyl groups, especially from 2 to 8 carbonyl groups. The bridging group optionally incorporates from 0 to 4 unsaturated carbon-carbon double bonds; and/or from 0 to 4 C₃ to C₁₀ aryl or heteroaryl groups in the chain. Optionally, the chain is interspersed with from 0 to 11, especially from 2 to 11, chain heteroatoms selected from N, O and S, with the proviso that no two heteroatoms are adjacent to one another. Advantageously, a chain carbon atoms in the bridging group is substituted with a pendant connecting group selected from amine, carboxy, alkyne, azide, hydroxyl or thiol. Advantageously the bridging group includes at least one amide linkage in the chain.”

Preferably the payload is a therapeutic agent, especially one of those mentioned above.

### The Protein

For convenience in this section and elsewhere, “protein” should be understood to include “protein and peptide” except where the context requires otherwise.

Suitable proteins which may be present in the conjugates of the invention include for example peptides, polypeptides, antibodies, antibody fragments, enzymes, cytokines, chemokines, receptors, blood factors, peptide hormones, toxin, transcription proteins, or multimeric proteins.

Enzymes include carbohydrate-specific enzymes, proteolytic enzymes and the like, for example the oxidoreductases, transferases, hydrolases, lyases, isomerases and ligases disclosed by U.S. Pat. No. 4,179,337. Specific enzymes of interest include asparaginase, arginase, adenosine deaminase, superoxide dismutase, catalase, chymotrypsin, lipase, uricase, bilirubin oxidase, glucose oxidase, glucuronidase, galactosidase, glucocerbrosidase, glucuronidase, and glutaminase.

Blood proteins include albumin, transferrin, Factor VII, Factor VIII or Factor IX, von Willebrand factor, insulin, ACTH, glucagen, somatostatin, somatotropins, thymosin, parathyroid hormone, pigmentary hormones, somatomedins, erythropoietin, luteinizing hormone, hypothalamic releasing factors, antidiuretic hormones, prolactin, interleukins, interferons, for example IFN-α or IFN-β, colony stimulating factors, hemoglobin, cytokines, antibodies, antibody fragments, chorionicgonadotropin, follicle-stimulating hormone, thyroid stimulating hormone and tissue plasminogen activator.

Other proteins of interest are allergen proteins disclosed by Dreborg et al Crit. Rev. Therap. Drug Carrier Syst. (1990) 6 315-365 as having reduced allergenicity when conjugated with a polymer such as poly(alkylene oxide) and consequently are suitable for use as tolerance inducers. Among the allergens disclosed are Ragweed antigen E, honeybee venom, mite allergen and the like.

Glycopolypeptides such as immunoglobulins, ovalbumin, lipase, glucocerebrosidase, lectins, tissue plasminogen activator and glycosylated interleukins, interferons and colony stimulating factors are of interest, as are immunoglobulins such as IgG, IgE, IgM, IgA, IgD and fragments thereof.

Of particular interest are receptor and ligand binding proteins and antibodies and antibody fragments which are used in clinical medicine for diagnostic and therapeutic purposes. Antibody-drug conjugates, especially where the drug is a cytotoxic drug, for example an auristatin or a maytansinoid, are an especially preferred embodiment of the invention.

The protein may be derivatised or functionalised if desired. In particular, prior to conjugation, the protein, for example a native protein, may have been reacted with various blocking groups to protect sensitive groups thereon; or it may have been previously conjugated with one or more polymers or other molecules. It may contain a polyhistidine tag, which during the conjugation reaction can be targeted by the conjugating reagent.

### Bonding of the Protein or Peptide, and Conjugating Reagents

The conjugating reagents of the invention are of the general type disclosed in WO 2005/007197 and WO 2010/000393. The functional groupings II and II′ are chemical equivalents of each other. When a reagent containing a group II reacts with a protein, a first leaving group L is lost to form in situ a conjugating reagent containing a group II′ which reacts with a first nucleophile. The second leaving group L is then lost, and reaction with a second nucleophile occurs. Thus as an alternative to using a reagent containing the functional grouping II as starting material, reagents containing the functional grouping II′ may be used as starting material.

A leaving group L may for example be —SP, —OP, —SO₂P, —OSO₂P, —N⁺PR²R³, halogen, or —OØ, in which P represents a hydrogen atom or an alkyl (preferably C₁₋₆alkyl), aryl (preferably phenyl), or alkyl-aryl (preferably C₁₋₆alkyl-phenyl) group, or is a group which includes a portion —(CH₂CH₂O)ₙ— in which n is a number of two or more, and each of R² and R³ independently represents a hydrogen atom, a C₁₋₄alkyl group, or a group P, and Ø represents a substituted aryl, especially phenyl, group, containing at least one electron withdrawing substituent, for example —CN, —NO₂, —CO₂R^{a}, —COH, —CH₂OH, —COR^{a}, —OR^{a}, —OCOR^{a}, —OCO₂R^{a}, —SR^{a}, —SOR^{a}, —SO₂R^{a}, —NHCO R^{a}, —NR^{a}, COR^{a}, —NHCO₂R^{a}, —NPCO₂R^{a}, —NO, —NHOH, —NR^{a} OH, —C═N—NHCOR^{a}, —C═N—NR^{a} COR^{a}, —N⁺R^{a}₃, —N⁺HR^{a}₂, —N⁺H₂R^{a}, halogen, especially chlorine or, especially, fluorine, —C≡CR^{a}, —C═CR^{a}₂ and —C═CHR^{a}, in which each R^{a} represents a hydrogen atom or an alkyl (preferably C₁₋₆alkyl), aryl (preferably phenyl), or alkyl-aryl (preferably C₁₋₆alkyl-phenyl) group.

Conjugating reagents in which P represents a group which includes a portion —(CH₂CH₂O)ₙ— in which n is a number of two or more are the subject of our copending application GB 1418186. This application discloses the following:
“The leaving group may for example include —(CH₂CH₂O)ₙ—R¹ where R¹ is a capping group. A very wide range of capping groups may be used. R¹ may for example be a hydrogen atom, an alkyl group, especially a C₁₋₄alkyl group, particularly a methyl group, or an optionally substituted aryl group, for example an optionally substituted phenyl group, for example a tolyl group. Alternatively, the capping group may include a functional group such as a carboxyl group or an amine group. Such capping groups may for example have the formula —CH₂CH₂CO₂H or —CH₂CH₂NH₂, and may be prepared by functionalising the terminal unit of a —(CH₂CH₂O)ₙ— chain. Alternatively, rather than being terminated by a capping group, the —(CH₂CH₂O)ₙ— group may have two points of attachment within the conjugating reagent such that chemically the equivalent of two leaving groups are present, capable of reacting with two nucleophiles.
The —(CH₂CH₂O)ₙ— portion of the leaving group is based on PEG, polyethylene glycol. The PEG may be straight-chain or branched, and it may be derivatised or functionalised in any way. n is a number of 2 or more, for example 2, 3, 4, 5, 6, 7, 8, 9 or 10. For example, n may be from 5 to 9. Alternatively, n may be a number of 10 or more. There is no particular upper limit for n. n may for example be 150 or less, for example 120 or less, for example 100 or less. For example n may be from 2 to 150, for example from 7 to 150, for example from 7 to 120. The PEG portion —(CH₂CH₂O)ₙ— of a leaving group may for example have a molecular weight of from 1 to 5 kDa; it may for example be 1 kDa, 2 kDa, 3 kDa, 4 kDa or 5 kDa. A leaving group may if desired contain two or more portions —(CH₂CH₂O)ₙ— separated by one or more spacers.
A leaving group in a conjugating reagent according to the invention is suitably of the formula —SP, —OP, —SO₂P, —OSO₂P, —N⁺PR²R³, in which P is a group which includes a portion —(CH₂CH₂O)ₙ— and each of R² and R³ independently represents a hydrogen atom, a C₁₋₄alkyl group, or a group P. Preferably each of R² and R³ represents a C₁₋₄alkyl group, especially a methyl group, or, especially, a hydrogen atom. Alternatively, the conjugating reagent may include a group of formula —S—P—S—; —O—P—O—; —SO₂—P—SO₂—; —OSO₂—P—OSO₂—; and —N⁺R²R³—P—N⁺R²R³—. Specific groups of this type include —S—(CH₂CH₂O)ₙ—S—, —O—(CH₂CH₂O)ₙ—O—; —SO₂—(CH₂CH₂O)ₙ—SO₂—; —OSO₂—(CH₂CH₂O)ₙ—OSO₂—; or —N⁺R²R³—(CH₂CH₂O)ₙ—N⁺R²R³—. They can also include groups of the type:

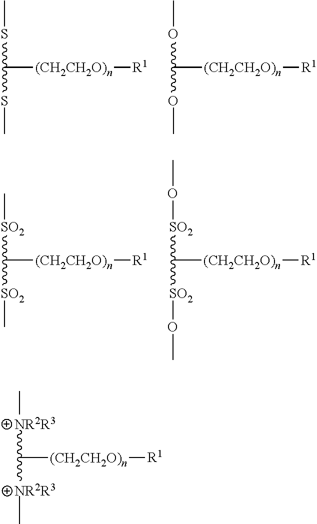

where the —(CH₂CH₂O)ₙ— group is carried by any suitable linking group, for example an alkyl group. These divalent groups are chemically equivalent to two leaving groups capable of reacting with two nucleophiles.”

An especially preferred leaving group L present in a novel conjugating reagent according to the present invention is —SP or —SO₂P, especially —SO₂P. Within this group, one preferred embodiment is where P represents a phenyl or, especially, a tosyl group. Another preferred embodiment is where P represents a group which includes a portion —(CH₂CH₂O)ₙ—.

The electron withdrawing group W may for example be a keto group —CO—, an ester group —O—CO— or a sulfone group —SO₂—. Preferably W′ represents one of these groups or a group obtainable by reduction of one of these groups as described below. Preferably W represents a keto group, and preferably W′ represents a keto group or a group obtainable by reduction of a keto group, especially a CH·OH group.

Preferably the groupings F′ and F have the formula:

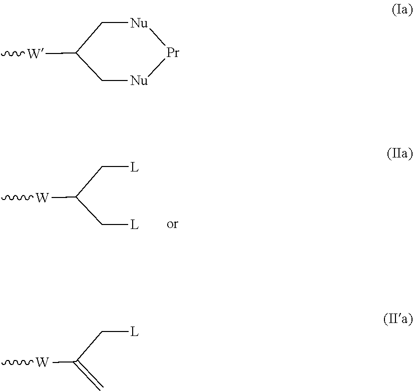

especially

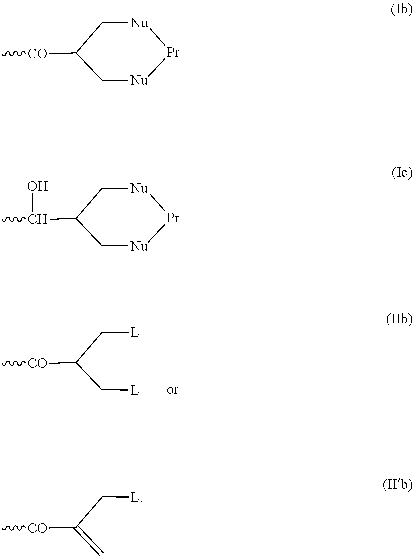

Nucleophilic groups in proteins are for example provided by cysteine, lysine or histidine residues, and Nu may for example be a sulfur atom or an amine group. In one preferred embodiment of the invention, each Nu represents a sulfur atom present in a cysteine residue present in the protein. Such structures may be obtained by reduction of a disulfide bond present in the protein. In another embodiment, each Nu represents an imidazole group present in a histidine residue present in a polyhistidine tag attached to said protein.

### Conjugating Processes

Conjugating reagents according to the invention may be reacted with a protein or peptide to form a conjugate according to the invention, and such a reaction forms a further aspect of the invention. Thus, a conjugating reagent including the functional grouping II or II′ is reacted with a protein or peptide to form a conjugate including the grouping I. The immediate product of the conjugation process is a conjugate which contains an electron-withdrawing group W. However, the conjugation process is reversible under suitable conditions. This may be desirable for some applications, for example where rapid release of the protein is required, but for other applications, rapid release of the protein may be undesirable. It may therefore be desirable to stabilise the conjugates by reduction of the electron-withdrawing moiety W to give a moiety which prevents release of the protein. Accordingly, the process described above may comprise an additional optional step of reducing the electron withdrawing group W in the conjugate. The use of a borohydride, for example sodium borohydride, sodium cyanoborohydride, potassium borohydride or sodium triacetoxyborohydride, as reducing agent is particularly preferred. Other reducing agents which may be used include for example tin(II) chloride, alkoxides such as aluminium alkoxide, and lithium aluminium hydride.

Thus, for example, a moiety W containing a keto group may be reduced to a moiety containing a CH(OH) group; an ether group CH·OR^{a} may be obtained by the reaction of a hydroxy group with an etherifying agent; an ester group CH·O·C(O)R^{a} may be obtained by the reaction of a hydroxy group with an acylating agent; an amine group CH·NH₂, CH·NHR^{a} or CH·NR^{a}₂ may be prepared from a ketone by reductive amination; or an amide CH·NHC(O)R^{a} or CH·N(C(O)R^{a})₂ may be formed by acylation of an amine. A sulfone may be reduced to a sulfoxide, sulfide or thiol ether.

A key feature of using conjugating reagents of the invention is that an α-methylene leaving group and a double bond are cross-conjugated with an electron withdrawing function that serves as a Michael activating moiety. If the leaving group is prone to elimination in the cross-functional reagent rather than to direct displacement and the electron-withdrawing group is a suitable activating moiety for the Michael reaction then sequential intramolecular bis-alkylation can occur by consecutive Michael and retro Michael reactions. The leaving moiety serves to mask a latent conjugated double bond that is not exposed until after the first alkylation has occurred to give a reagent including the functional grouping II′ and bis-alkylation results from sequential and interactive Michael and retro-Michael reactions. The cross-functional alkylating agents may contain multiple bonds conjugated to the double bond or between the leaving group and the electron withdrawing group.

Where bonding to the protein is via two sulfur atoms derived from a disulfide bond in the protein, the process may be carried out by reducing the disulfide bond following which the reduced product reacts with the reagent according to the invention. Preferably the disulfide bond is reduced and any excess reducing agent is removed, for example by buffer exchange, before the conjugating reagent is introduced. The disulfide bond can be reduced, for example, with dithiothreitol, mercaptoethanol, or tris-carboxyethylphosphine using conventional methods.

Conjugation reactions may be carried out under similar conditions to known conjugation processes, including the conditions disclosed in WO 2005/007197, WO 2009/047500, WO 2014/064423 and WO 2014/064424. The process may for example be carried out in a solvent or solvent mixture in which all reactants are soluble. For example, the protein may be allowed to react directly with the polymer conjugating reagent in an aqueous reaction medium. This reaction medium may also be buffered, depending on the pH requirements of the nucleophile. The optimum pH for the reaction will generally be at least 4.5, typically between about 5.0 and about 8.5, preferably about 6.0 to 7.5. The optimal reaction conditions will of course depend upon the specific reactants employed.

Reaction temperatures between 3-40° C. are generally suitable when using an aqueous reaction medium. Reactions conducted in organic media (for example THF, ethyl acetate, acetone) are typically conducted at temperatures up to ambient. In one preferred embodiment, the reaction is carried out in aqueous buffer which may contain a proportion of organic solvent, for example up to 20% by volume of organic solvent, typically from 5 to 20% by volume of organic solvent.

The protein can be effectively conjugated using a stoichiometric equivalent or a slight excess of conjugating reagent. However, it is also possible to conduct the conjugation reaction with an excess stoichiometry of conjugating reagent, and this may be desirable for some proteins. The excess reagent can easily be removed, for example by ion exchange chromatography or HPLC, during subsequent purification of the conjugate.

Of course, it is possible for more than one conjugating reagent to be conjugated to a protein, where the protein contains sufficient suitable attachment points. For example, in a protein which contains two different disulfide bonds, or in a protein which contains one disulfide bond and also carries a polyhistidine tag, it is possible to conjugate two molecules of the reagent per molecule of protein, and such conjugates form part of the present invention.

### The Linker

The linker which connects the therapeutic, diagnostic or labelling agent to the protein or peptide bonding portion in the conjugates of the invention or to the functional grouping in conjugating reagents of the invention must include one or more PEG portions as described above. It may also contain any other desired groups, particularly any of the conventional groups commonly found in this field.

Subsection (i). In one embodiment, the linker between the payload and the grouping of formula F′/F, and particularly that portion of the linker immediately adjacent the grouping of formula F′/F, may include an alkylene group (preferably a C₁₋₁₀ alkylene group), or an optionally-substituted aryl or heteroaryl group, any of which may be terminated or interrupted by one or more oxygen atoms, sulfur atoms, —NR^{a} groups (in which R^{a} represents a hydrogen atom or an alkyl (preferably C₁₋₆alkyl), aryl (preferably phenyl), or alkyl-aryl (preferably C₁₋₆alkyl-phenyl) group), keto groups,

—O—CO— groups, —CO—O— groups, —O—CO—O, —O—CO—NR^{a}—, —NR—CO—O—, —CO—NR^{a}— and/or —NR^{a}·CO— groups. Suitable aryl groups include phenyl and naphthyl groups, while suitable heteroaryl groups include pyridine, pyrrole, furan, pyran, imidazole, pyrazole, oxazole, pyridazine, pyrimidine and purine. Especially suitable linking groups are heteroaryl or, especially, aryl groups, especially phenyl groups. These may be adjacent a further portion of the linking group which is, or contains, a —NR^{a}·CO— or —CO·NR^{a}— group, for example an —NH·CO— or —CO·NH— group. Here and elsewhere throughout this Specification, where a group R^{a} is present, this is preferably a C₁₋₄alkyl, especially a methyl group or, especially, a hydrogen atom.

Substituents which may be present on an optionally substituted aryl, especially phenyl, or heteroaryl group include for example one or more of the same or different substituents selected from alkyl (preferably C₁₋₄alkyl, especially methyl, optionally substituted by OH or CO₂H), —CN, —NO₂, —CO₂R^{a}, —COH, —CH₂OH, —COR^{a}, —OR^{a}, —OCOR^{a}, —OCO₂R^{a}, —SR^{a}, —SOR^{a}, —SO₂R^{a}, —NHCOR^{a}, —NR^{a}COR^{a}, —NHCO₂R^{a}, —NR^{a}·CO₂R^{a}, —NO, —NHOH, —NR^{a}·OH, —C═N—NHCOR^{a}, —C═N—NR^{a}·COR^{a}, —N⁺R^{a}₃, —N⁺H₃, —N⁺HR^{a}₂, —N⁺H₂R^{a}, halogen, for example fluorine or chlorine, —C═CR^{a}, —C═CR^{a}₂ and —C═CHR^{a}, in which each R^{a} independently represents a hydrogen atom or an alkyl (preferably C₁₋₆alkyl), aryl (preferably phenyl), or alkyl-aryl (preferably C₁₋₆alkyl-phenyl) group. The presence of electron withdrawing substituents is especially preferred. Preferred substituents include for example CN, NO₂, —OR^{a}, —OCOR^{a}, —SR^{a}, —NHCOR^{a}, —NR^{a}·COR^{a}, —NHOH and —NR^{a}·COR^{a}.

Preferably the linker includes one of the above groups adjacent the grouping F′/F. Especially preferred are conjugates and conjugating reagents which include the grouping:

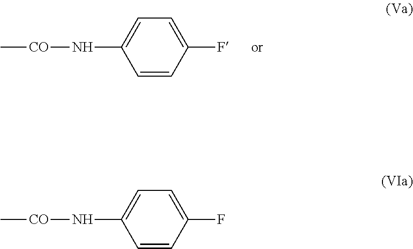

or, especially:

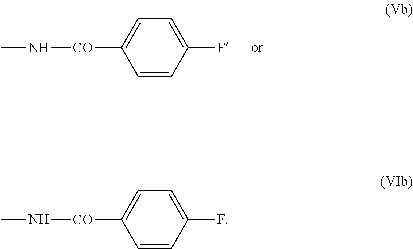

In the above formulae, preferably F′ has the formula Ia or Ib above, and preferably F has the formula IIa, II′a, IIb or II′b above.

Subsection (ii). In one embodiment, the linker may contain a degradable group, i.e. it may contain a group which breaks under physiological conditions, separating the payload from the protein to which it is, or will be, bonded. Alternatively, it may be a linker that is not cleavable under physiological conditions. Where a linker breaks under physiological conditions, it is preferably cleavable under intracellular conditions. Where the target is intracellular, preferably the linker is substantially insensitive to extracellular conditions (i.e. so that delivery to the intracellular target of a sufficient dose of the therapeutic agent is not prohibited).

Where the linker contains a degradable group, this is generally sensitive to hydrolytic conditions, for example it may be a group which degrades at certain pH values (e.g. acidic conditions). Hydrolytic/acidic conditions may for example be found in endosomes or lysosomes. Examples of groups susceptible to hydrolysis under acidic conditions include hydrazones, semicarbazones, thiosemicarbazones, cis-acotinic amides, orthoesters and ketals. Examples of groups susceptible to hydrolytic conditions include:

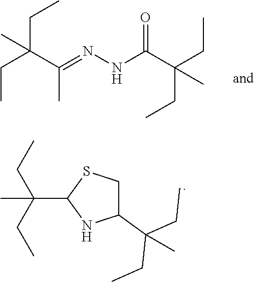

In a preferred embodiment, the linker includes

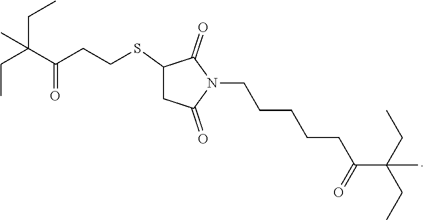 [SMILES: CCC(C)(CC)C(=O)CCCCCN1C(=O)CC(SCCC(=O)C(C)(CC)CC)C1=O]

For example, it may include:

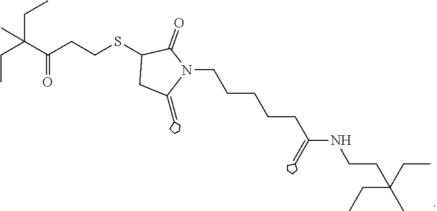 [SMILES: CCC(C)(CC)CCNC(CCCCCN1C(=O)C(SCCC(=O)C(C)(CC)CC)CC1=C1CCCC1)=C1CCCC1]

The linker may also be susceptible to degradation under reducing conditions. For example, it may contain a disulfide group that is cleavable on exposure to biological reducing agents, such as thiols. Examples of disulfide groups include:

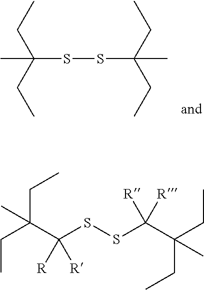

in which R, R′, R″ and R″ are each independently hydrogen or C₁₋₄alkyl. In a preferred embodiment the linker includes

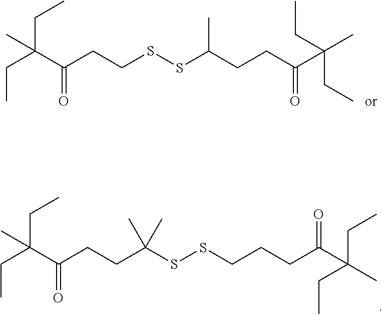

For example, it may include

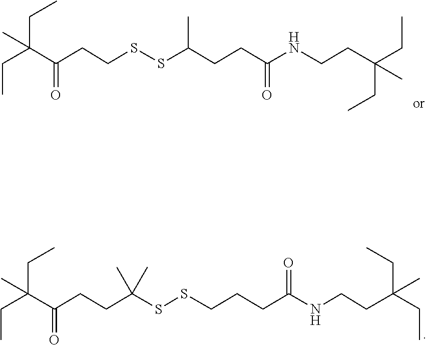

The linker may also contain a group which is susceptible to enzymatic degradation, for example it may be susceptible to cleavage by a protease (e.g. a lysosomal or endosomal protease) or peptidase. For example, it may contain a peptidyl group comprising at least one, for example at least two, or at least three amino acid residues (e.g. Phe-Leu, Gly-Phe-Leu-Gly, Val-Ala, Val-Cit, Phe-Lys). For example, it may include an amino acid chain having from 1 to 5, for example 2 to 4, amino acids. Another example of a group susceptible to enzymatic degradation is:

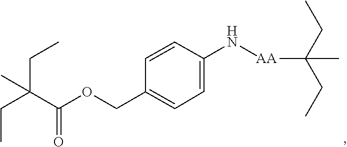

wherein AA represents a protease-specific amino acid sequence, such as Val-Cit.

In a preferred embodiment, the linker includes:

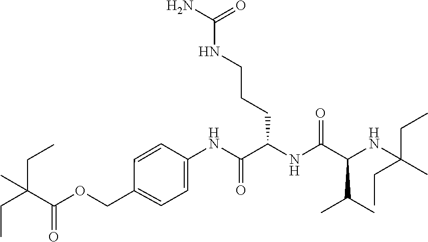 [SMILES: CCC(C)(CC)N[C@H](C(=O)N[C@@H](CCCNC(N)=O)C(=O)Nc1ccc(COC(=O)C(C)(CC)CC)cc1)C(C)C]

For example, it may include

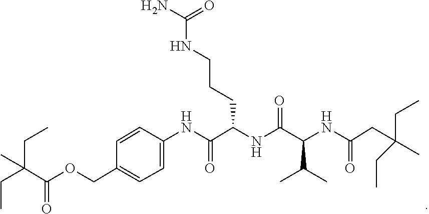 [SMILES: CCC(C)(CC)CC(=O)N[C@H](C(=O)N[C@@H](CCCNC(N)=O)C(=O)Nc1ccc(COC(=O)C(C)(CC)CC)cc1)C(C)C]

The linker may carry a single payload D, or more than one group D. Multiple groups D may be incorporated by the use of a branching linker, which may for example incorporate an aspartate or glutamate or similar residue. This introduces a branching element of formula:

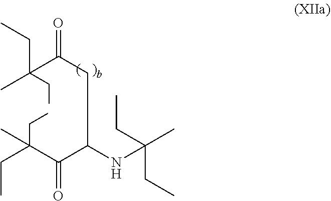

where b is 1, 2 or 3, b=1 being aspartate and b=2 being glutamate, and b=3 representing one preferred embodiment. Each of the acyl moieties in the above formula may be coupled to a group D. The branching group above may incorporate a —CO·CH₂— group, thus:

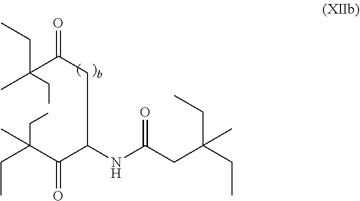

If desired, the aspartate or glutamate or similar residue may be coupled to further aspartate and/or glutamate and/or similar residues, for example:

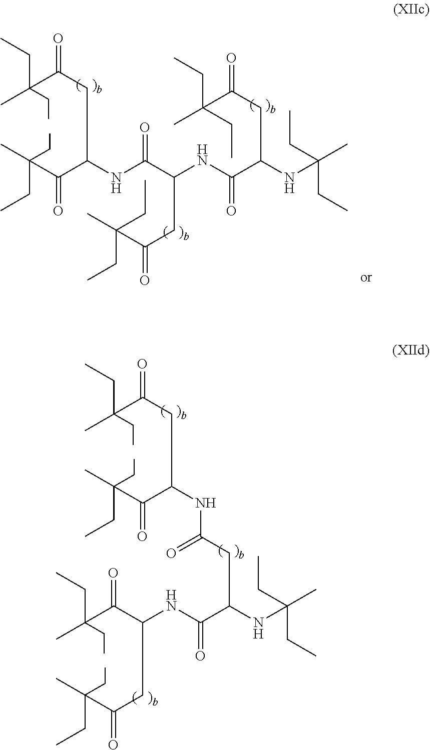

and so on.

As will be apparent, many alternative configurations for the linker between the grouping F′/F and the payload are possible. One preferred configuration may be represented schematically as follows:

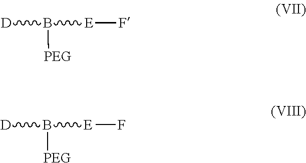

in which E represents one of the groups mentioned in subsection (i) above, and B represents one of the groups mentioned in this subsection (ii).

A specific, particularly preferred construction is shown below:

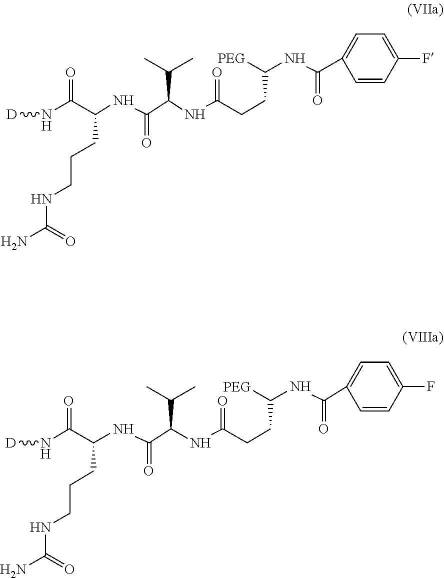

in which F′ and F have the meanings given above.

Subsection (iii). The linker which connects the therapeutic, diagnostic or labelling agent to the protein or peptide bonding portion in the conjugates of the invention or to the functional grouping in the conjugating reagents of the invention may contain additional PEG in addition to the pendant PEG chain which has a terminal end group of formula —CH₂CH₂OR. It may for example contain PEG in the backbone of the linker, shown schematically thus:

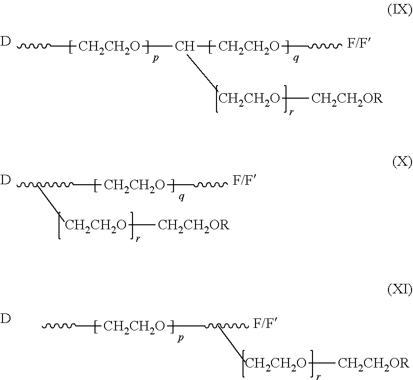

In these formulae, p, q and r represent the number of PEG units present in the various PEG chains present in the linker of the conjugate or the reagent. For clarity, the PEG units are shown as straight-chain units, but it will be understood that any of the units may include branched chains.

Subsection (iv). The linker which connects the therapeutic, diagnostic or labelling agent to the protein or peptide bonding portion in the conjugates of the invention or to the functional grouping in the conjugating reagents of the invention may contain two or more pendant PEG chains. This may be illustrated schematically for two pendant PEG chains thus:

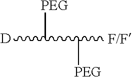

and obviously more than two pendant PEG chains may similarly be present. The linker may or may not contain additional PEG in addition to the pendant PEG chains, as described in subsection (iii) above.

Multiple pendant PEG chains may be incorporated into the linker using any suitable method. A pendant PEG chain may for example be introduced by reaction with any reactive grouping present in any of the linker portions discussed above. Branching groups of the formulae (XIIa-d) as described above may be used. For example, in one specific embodiment, two pendant PEG portions may be incorporated by use of a structure (XIIa):

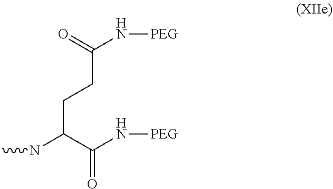

Alternatively, branching may be introduced by use of a polyol functionality, for example:

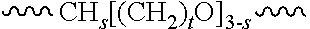

in which s is 0, 1 or 2, and t is 1 to 4. For example, in one specific embodiment, three pendant PEG portions may be incorporated by use of a structure:

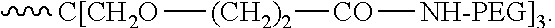

### BRIEF DESCRIPTION OF THE DRAWINGS

FIGS. 1 and 2 show the results of Example 6.

FIG. 3 shows the results of Example 7.

FIGS. 4a, 4b and 4c show the results of Example 14.

FIGS. 5a and 5b show the results of Example 15.

FIGS. 6a, 6b, 6c and 6d show the results of Example 16.

FIGS. 7a, 7b and 7c show the results of Example 17.

FIGS. 8a and 8b show the results of Example 18.

FIGS. 9a and 9b show the results of Example 19.

FIG. 10 shows the results of Example 23.

FIG. 11 shows the results of Example 25.

The following Examples illustrate the invention.

### Example 1: Synthesis of Conjugation Reagent 1 Comprising an Auristatin Cytotoxic Payload

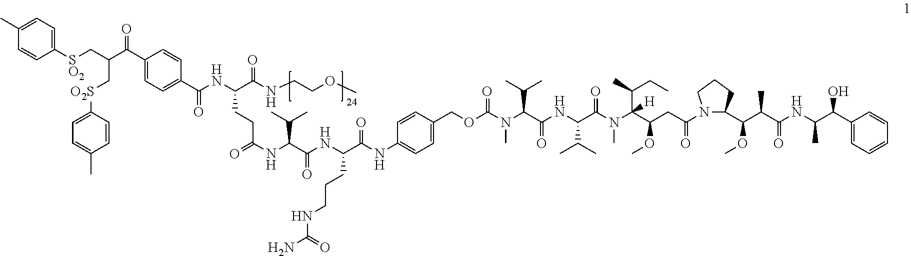

### Step 1: Synthesis of Compound 2.

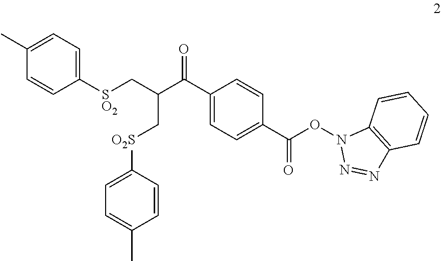 [SMILES: Cc1ccc(S(=O)(=O)CC(CS(=O)(=O)c2ccc(C)cc2)C(=O)c2ccc(C(=O)On3nnc4ccccc43)cc2)cc1]

A solution of 4-[2,2-bis[(p-tolylsulfonyl)-methyl]acetyl]benzoic acid (1.0 g, Nature Protocols, 2006, 1(54), 2241-2252) was added to N-hydroxybenzotriazole hydrate (306 mg) in anhydrous THF (10 mL) under a nitrogen atmosphere. The resulting solution was cooled to 0° C. and diisopropylcarbodiimide (310 μL) was added dropwise. The reaction mixture was stirred for 20 min at 0° C. before being warmed to room temperature. Additional THF (10 mL) was added to the reaction mixture after 1 h. After 18 h, the formed precipitate was filtered and washed with cold THF (2×5 mL) before being dried in vacuo. The solid was stirred with MeOH (10 mL) for 1 h at room temperature, collected by filtration and washed sequentially with MeOH (2×5 mL) and Et₂O (5 mL). The solid was then dried in vacuo to give bis-tolylsulfonyl-propanoyl-benzoic HOBt ester compound 2 as a white solid (1.1 g, 88%). m/z [M+H]⁺ (618, 100%).

### Step 2: Synthesis of Compound 3.

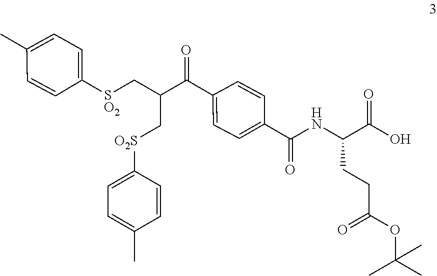 [SMILES: Cc1ccc(S(=O)(=O)CC(CS(=O)(=O)c2ccc(C)cc2)C(=O)c2ccc(C(=O)N[C@@H](CCC(=O)OC(C)(C)C)C(=O)O)cc2)cc1]

To a stirred suspension of (S)-Glu-5-(OtBu) (198 mg) in anhydrous DMF (20 mL) under a nitrogen atmosphere was added N-methylmorpholine (NMM) (107 μL). The reaction mixture was cooled to 0° C. before compound 2 (603 mg) was added. The resulting suspension was stirred at 0° C. for 1 h, after which the reaction mixture was allowed to warm to room temperature. After 19 h, the resulting solution was concentrated in vacuo and purified by reverse phase column C18-column chromatography eluting with buffer A (v/v): water:5% acetonitrile:0.1% formic acid and buffer B (v/v): acetonitrile:0.1% formic acid (100:0 v/v to 0:100 v/v). The organic solvent was removed in vacuo and the aqueous solvent was removed by lyophilisation to give the bis-tolylsulfonyl-propanoyl-benzamide-L-Glu-[O^{t}Bu]-[OH] compound 3 as a white solid (198 mg, 67%). ¹H NMR (400 MHZ, CDCl₃) δ 7.98 (1H, d), 7.86 (2H), 7.71-7.65 (6H, m), 7.36 (4H, d), 4.68 (1H, ddd), 4.34 (1H, q), 3.62 (2H, ddd), 3.50 (2H, ddd), 2.69 (1H ddd), 2.55-2.45 (1H, m), 2.48 (6H, s), 2.34-2.16 (2H, m), 1.46 (9H, s); m/z [2M+H]⁺ (1371, 74%), [2M-^{t}Bu]⁺ (1315, 70%), [M-^{t}Bu]⁺ (630, 100%).

### Step 3: Synthesis of Compound 4.

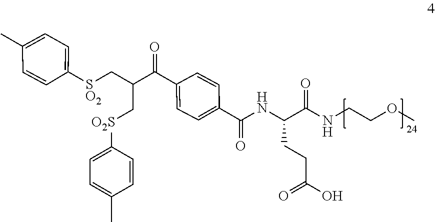

Compound 3 (50 mg) and (benzotriazol-1-yloxy)tris-(dimethylamino) phosphonium hexafluorophosphate (BOP) (40 mg) were dissolved in anhydrous DMF (3 mL), cooled to 0° C. and added to a solution of NH₂-PEG(24u)-OMe (99 mg) and NMM (10 μL) in anhydrous DMF (2 mL). The reaction mixture stirred at 0° C. and after 4 h, additional amounts of BOP (10 mg) and NMM (2.5 μL) were added to the reaction mixture and incubated for a further 15 min., before being stored at −20° C. for 18 h. The resultant reaction mixture was concentrated in vacuo and purified by reverse phase column C18-column chromatography, eluting with buffer A (v/v): water:5% acetonitrile:0.1% formic acid and buffer B (v/v): acetonitrile:0.1% formic acid (100:0 v/v to 0:100 v/v). The organic solvent was removed in vacuo and the aqueous solvent removed by lyophilisation to give bis-tolylsulfonyl-propanoyl-benzamide-L-Glu-[O^{t}Bu]-[PEG(24u)-OMe] as a colourless oil (128 mg, 100%). m/z [M+H]⁺ (1757, 100%), [M+2H]²⁺ (879, 100%). Bis-tolylsulfonyl-propanoyl-benzamide-L-Glu-[O^{t}Bu]-[PEG(24u)-OMe] (126.5 mg) was dissolved in formic acid (2.5 mL) and stirred under a nitrogen atmosphere at room temperature. After 20 h, the reaction mixture was concentrated in vacuo and dried under high vacuum for 18 h to give bis-tolylsulfonyl-propanoyl-benzamide-L-Glu-[OH]-[PEG(24u)-OMe] compound 4 as a colourless oil (122 mg, assumed quantitative yield). m/z [M+Na]⁺ (1723, 15%), [M+H]⁺ (1700, 100%). This material was used without any further purification.

### Step 4: Synthesis of Reagent 1.

A solution of compound 4 (13.0 mg), HATU (4.1 mg), val-cit-PAB-MMAE TFA salt (9.0 mg) in DMF (1.0 mL) under an argon atmosphere was cooled to 0° C. To this was added NMM (2.0 μL). After 1 h, an additional amount of HATU (4.1 mg) and NMM (2 μL) was added, and after a further 1.5 h the solution was stored at −20° C. for 72 h. The reaction solution was concentrated in vacuo, dissolved in acetonitrile (1.0 ml) and purified by reverse phase C18-column chromatography eluting with buffer A (v/v): water:5% acetonitrile:0.05% trifluoroacetic acid and buffer B (v/v): acetonitrile:0.05% trifluoroacetic acid (100:0 v/v to 0:100 v/v). The organic solvent was removed in vacuo and the aqueous solvent was removed by lyophilisation to give bis-tolylsulfonyl-propanoyl-benzamide-Glu-[NH-PEG(24u)-OMe]-[val-cit-PAB-MMAE] reagent 1 as a thick clear colourless oil (11.4 mg, 56%). m/z [M+H]⁺ (2805, 20%), [M+2H]²⁺ (1403, 75%), [M+3H]³⁺ (936, 100%).

### Example 2: Synthesis of Conjugation Reagent 5 Comprising a Maytansinoid Cytotoxic Payload

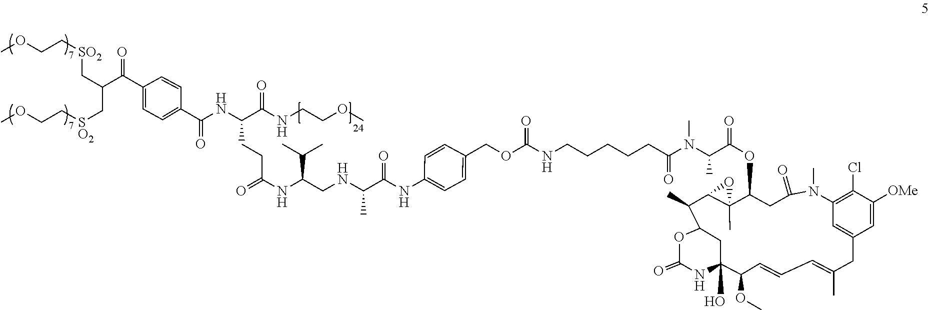

### Step 1: Synthesis of Compound 6.

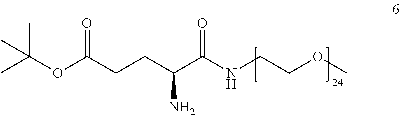

A solution of Fmoc-L-Glu-(OtBu)-OH (36 mg) in DMF (2 mL) was cooled to 0° C. under an argon atmosphere and (benzotriazol-a-yloxy)tris-(dimethylamino)phosphonium hexafluorophosphate BOP (41 mg) was added, followed by NH₂-PEG(24u)-OMe (100 mg) and N,N-diisopropylethylamine (19 μL). The solution was allowed to warm to room temperature and after 22 h the volatiles were removed in vacuo. The resulting residue was dissolved in dichloromethane (1 mL) and purified by normal phase column chromatography eluting with dichloromethane:methanol (100:0 v/v to 80:20 v/v). The organic solvent was removed in vacuo to give Fmoc-L-Glu-[O^{t}Bu]-[PEG(24u)-OMe] as a colourless oil (84 mg, 67%). Piperidine (49 μL) was added to a solution of compound Fmoc-L-Glu-[O^{t}Bu]-[PEG(24u)-OMe] (74 mg) in DMF (2 mL) under an argon atmosphere and the resulting solution stirred at room temperature for 22 h, after which the volatiles were removed in vacuo. The resulting residue was triturated with hexane (3×0.7 mL). The organic solvent was decanted each time and the resulting residue dried in vacuo to give the L-Glu-[OtBu]-[PEG(24u)-OMe] compound 6 as a white solid (61 mg, 97%). m/z [M+H]⁺ (1097, 10%), [M+2H]²⁺ (1035, 100%).

### Step 2: Synthesis of Compound 7.

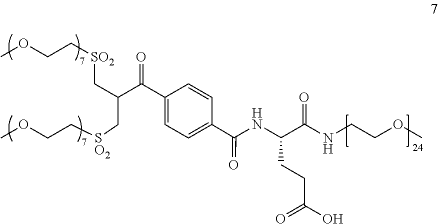

A solution of compound 6 (26.6 mg) in DMF (550 μL) was cooled to 0° C. under an argon atmosphere to which HATU (10.5 mg) was added and the solution stirred for 0.5 h at 0° C. To this was added a solution of 4-[2,2-bis[alpha-methoxy-omega-sulfonyl hepta(ethylene glycol)]acetyl]benzoic acid (32 mg, prepared in an analogous way to 4-[2,2-bis[(p-tolylsulfonyl)-methyl]acetyl]benzoic acid in Nature Protocols, 2006, 1(54), 2241-2252, but using alpha-methoxy-omega-mercapto hepta(ethylene glycol) instead of 4-methylbenzenethiol) in DMF (550 μL). The resulting solution was stirred for 5 min at 0° C. before addition of NMM (2.9 μL) and HATU (10.5 mg). The reaction solution was allowed to stir at 0° C. for 2 h before being warmed to room temperature and stirred for a further 3.5 h. After this time the volatiles were removed in vacuo. The resulting residue was dissolved in water and acetonitrile (v/v; 1/1, 1.2 ml), and purified by reverse phase C18-column chromatography eluting with buffer A (v/v): water:5% acetonitrile:0.1% formic acid and buffer B (v/v): acetonitrile:0.1% formic acid (100:0 v/v to 0:100 v/v). The organic solvent was removed in vacuo and the aqueous solvent removed by lyophilisation to give bis-mPEG(7u)sulfone-propanoyl-benzamide-L-Glu-[OtBu]-[PEG(24u)-OMe] as a colourless oil (30.5 mg, 55%). ¹H NMR (400 MHZ, MeOH-δ₄) 8.19 (2H, d), 8.04 (2H, d), 4.83-4.71 (1H, m), 4.58 (1H, dd), 3.92-3.83 (6H, m), 3.78-3.56 (140H, m), 3.57-3.51 (6H, m), 3.40 (4H, dd), 3.36 (3H, s), 3.35 (6H, s), 2.41 (2H, t), 2.24-2.13 (1H, m), 2.10-1.98 (1H, m), 1.45 (9H, s); m/z [M+Na]⁺ (2243, 50%), [M+H]⁺ (2221, 40%), [M+Na+2H]³⁺ (747,100%). A solution of bis-mPEG(7u)sulfone-propanoyl-benzamide-L-Glu-[OtBu]-[PEG(24u)-OMe] (30 mg) in dichloromethane (2 mL) under an argon atmosphere was cooled to 0° C. to which trifluoroacetic acid (500 μL) was added and the resulting solution stirred for 1.5 h. The reaction mixture was allowed to warm to room temperature and stirred for a further 1 h. After this time the volatiles were removed in vacuo. The resulting residue was dissolved in water and acetonitrile (v/v; 1/1, 0.6 mL), and purified by reverse phase C18-column chromatography eluting with buffer A (v/v): water:5% acetonitrile:0.05% trifluoroacetic acid and buffer B (v/v): acetonitrile:0.05% trifluoroacetic acid (100:0 v/v to 0:100 v/v). The organic solvent was removed in vacuo and the aqueous solvent removed by lyophilisation to give the bis-mPEG(7u)sulfone-propanoyl-benzamide-L-Glu-[OH]-[PEG(24u)-OMe] compound 7 as a colourless oil (20 mg, 68%). 1H NMR (400 MHZ, MeOH-δ₄) 8.19 (2H, d), 8.04 (2H, d), 4.81-4.72 (1H, m), 4.59 (1H, dd), 3.92-3.84 (6H, m), 3.67-3.50 (146H, m), 3.40 (4H, dd), 3.36 (3H, s), 3.35 (6H, s), 2.48 (2H, t), 2.26-2.15 (1H, m), 2.15-2.03 (1H, m); m/z [M+H]⁺ (2165, 55%), [M+2H]²⁺ (1083, 60%), [M+2H+Na]³⁺ (729, 100%).

### Step 3: Synthesis of Reagent 5

A solution of compound 7 (15.0 mg) in DMF (600 μL) was cooled to 0° C. under an argon atmosphere. HATU (2.9 mg) was added and the solution stirred for 0.5 h at 0° C. To this was added a solution of val-ala-PAB-AHX-DM1 (9.2 mg) and NMM (0.8 μL) in DMF (600 μL), which had been stirred at room temperature for 0.5 h. After 5 min, an additional amount of HATU (2.9 mg) and NMM (0.8 μL) was added and the reaction mixture stirred at 0° C. After 3 h, an additional amount of HATU (0.7 mg) was added and the reaction mixture stirred at 0° C. After a further 2 h, the reaction was stored at −20° C. for 16 h. The reaction solution was concentrated in vacuo and purified by reverse phase C18-column chromatography eluting with buffer A (v/v): water:5% acetonitrile:0.05% trifluoroacetic acid and buffer B (v/v): acetonitrile:0.05% trifluoroacetic acid (100:0 v/v to 0:100 v/v). The organic solvent was removed in vacuo and the aqueous solvent removed by lyophilisation to give the bis-mPEG(7u)sulfone-propanoyl-benzamide-L-Glu-[val-ala-PAB-AHX-DM1]-[PEG(24u)-OMe] compound 5 as a thick clear colourless oil (14.3 mg, 64%). ¹H NMR (600 MHZ, MeOH δ₄) (selected characteristic signals) 5.69 (1H, dd), 6.59 (1H, dd), 6.68 (1H, s), 6.69 (1H, d), 7.10 (1H, s), 7.28 (2H, d), 7.57 (2H, d), 8.01 (2H, d), 8.16 (2H, d); m/z [M-AHX-DM1]⁺ (2422, 40%).

### Example 3: Synthesis of a Conjugation Reagent 8 Comprising 7 Repeat Unit Polymeric Leaving Groups and a Maytansinoid Cytotoxic Payload

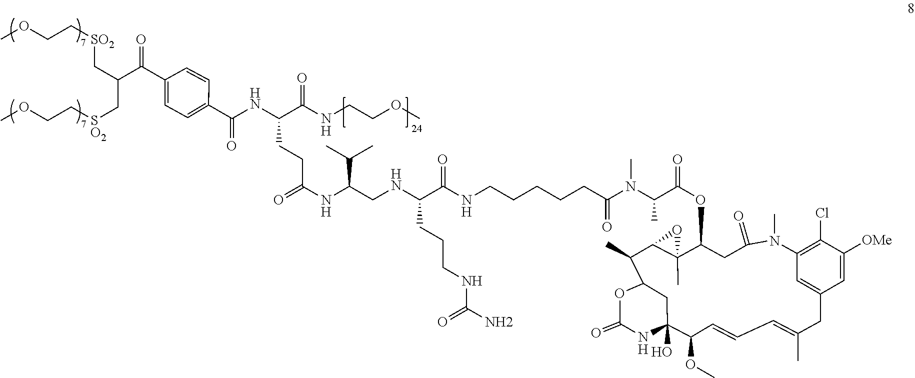

A solution of compound 7 (12.4 mg) in DMF (500 μL) was cooled to 0° C. under an argon atmosphere. HATU (2.4 mg) was added and the solution stirred for 0.5 h at 0° C. To this was added a solution of val-cit-AHX-DM1 made in an analogous way to compound 10A (6.4 mg) and NMM (0.7 μL) in DMF (500 μL), which had been stirred at room temperature for 0.5 h. After 5 min, an additional amount of HATU (1.2 mg) and NMM (0.4 L) was added and the reaction mixture stirred at room temperature. After 2 h, an additional amount of HATU (1.2 mg) and NMM (0.4 μL) was added and the reaction mixture stirred at room temperature. After a further 1 h, the reaction solution was concentrated in vacuo and purified by reverse phase C18-column chromatography eluting with buffer A (v/v): water:5% acetonitrile:0.05% trifluoroacetic acid and buffer B (v/v): acetonitrile:0.05% trifluoroacetic acid (100:0 v/v to 0:100 v/v). The organic solvent was removed in vacuo and the aqueous solvent removed by lyophilisation to give bis-mPEG(7u)sulfone-propanoyl-benzamide-L-Glu-[val-cit-AHX-DM1]-[PEG(24u)-OMe] 8 as a thick clear colourless oil (9.6 mg, 53%). m/z [M−H₂O]⁺ (3148, 8%), [M−H₂O]²⁺ (1575, 40%), [M−H₂O]³⁺ (1050, 100%), 1036 [M−NHCO—H₂O]³⁺.

### Example 4: Synthesis of a Conjugation Reagent 2 Comprising an Auristatin Cytotoxic Payload

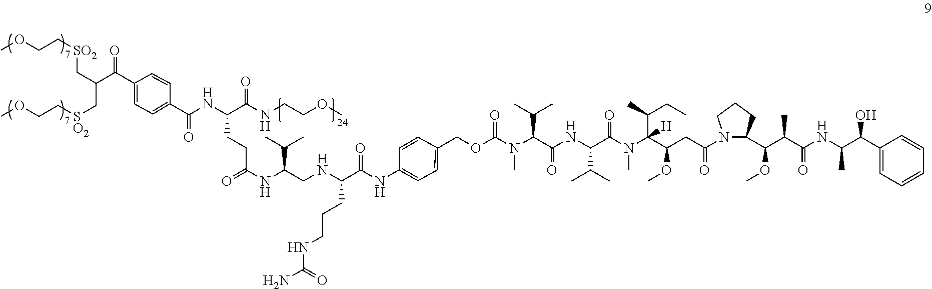

Reagent 2 was synthesised in analogous way to reagent 8 of Example 3 from compound 7 and val-cit-PAB-MMAE TFA salt. Bis-mPEG(7u)sulfone-propanoyl-benzamide-L-Glu-[val-cit-PAB-MMAE]-[PEG(24u)-OMe] 2 was isolated as a colourless oil. m/z [M+H]⁺ (3270, 12%), [M+2H]²⁺ (1636, 50%), [M+3H]³⁺ (1091, 100%).

### Example 5. Preparation of Antibody Drug Conjugates

Antibody drug conjugates were prepared by methods analogous to those described in WO2014064423 and WO2014064424. Briefly, antibody (trastuzumab or brentuximab) was reduced using tris(2-carboxyethyl)phosphine at 40° C. for 1 h. Conjugation of the antibody with 1.5 molar equivalents of reagent (i.e., 1, 5, 8, 9) per inter-chain disulfide bond was then performed by dissolving reagents to a final concentration of 1.6 mM in either acetonitrile or DMF. The antibody solution was diluted to 4.21 mg/mL with 20 mM sodium phosphate buffer, 150 mM NaCl, 20 mM EDTA, pH 7.5. Reagents were added to antibody and the final antibody concentration in the reaction was adjusted to 4 mg/mL with 20 mM sodium phosphate buffer, 150 mM NaCl, 20 mM EDTA, pH 7.5. Each solution was mixed gently and incubated at 22° C. Antibody drug conjugate product was purified by hydrophobic interaction chromatography for each conjugate.

### Example 6: In Vitro Cytotoxicity Comparison of Brentuximab Drug Conjugate 10 Prepared from Reagent 2 with the Brentuximab Drug Conjugate 11 Produced Using Reagent Bis-Tolylsulfonyl-Propanoyl-Benzamide-PEG(24u)-Val-Cit-PAB-MMAE, 12 Using the Method Described in WO2014064423

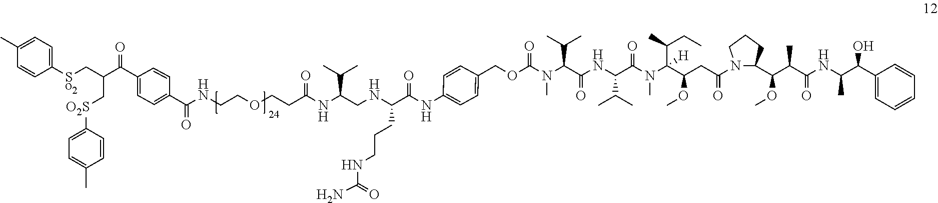

Purified brentuximab drug conjugates 10 and 11, with a drug to antibody ratio (DAR) of four as described in Example 6, were evaluated in vitro by measuring the inhibitory effect on cell growth of (CD30)-positive cell line Karpas 299 using the method described below.

Loss of tumour cell viability following treatment with cytotoxic drugs or ADCs in vitro can be measured by growing cell lines in the presence of increasing concentrations of drugs or ADCs and quantifying the loss of proliferation or metabolic activity using CellTiter Glo® Luminescence reagent (Promega Corp. Technical Bulletin TB288; Lewis Phillips G. D, Cancer Res 2008; 68:9280-9290). The protocol describes cell seeding, drug treatment and determination of the cell viability in reference to untreated cells based on ATP synthesis, which is directly related to the number of cells present in the well.

The human T cell lymphoma cell line Karpas 299 was obtained from Dr Abraham Karpas at the University of Cambridge. The cells were grown in RPMI medium (Life Technologies®), 10% fetal bovine serum, 100 u/mL Penicillin and 100 μg/mL Streptomycin.

CD30-positive Karpas 299 were counted using a Neubauer haemocytometer and adjusted to a cell density of 5×10⁴/mL. Cells were seeded (50 μL/well) into opaque-walled 96-well plates and incubated for 24 h at 37° C. and 5% CO₂.

Methods for cell culture were derived from product information sheet from supplier and references quoted therein, for example, Culture of Animal Cells: A Manual of Basic Technique by R. Ian Freshney 3^{rd} edition, published by Alan R. Liss, N.Y. 1994, or 5th edition published by Wiley-Liss, N.Y. 2005. Serial dilutions of ADC or free drug (MMAE), were made in triplicate by pipetting across a 96 well plate from columns 2-11 with 2-fold dilutions using the relevant cell culture medium as a diluent. The CD30-positive Karpas 299 were treated with drug concentrations shown in Table 1. Cells were then incubated with the drug at 37° C. and 5% CO₂ for a further 72 h.

TABLE 1

| | | Concentration |
|---|---|---|
| Cell line | Drug/drug-conjugate | range |
| Karpas 299 | MMAE (free drug) | 2500 pM-4.9 pM |
| Karpas 299 | Brentuximab-drug conjugate 10, | 333 pM-0.65 pM |
| | DAR4 | |
| Karpas 299 | Brentuximab-drug conjugate 11, | 333 pM-0.65 pM |
| | DAR4 | |

The cell viability assay was carried out using the Cell-Titer Glo® Luminescence reagent, as described by the manufacturer's instructions, (Promega Corp. Technical Bulletin TB288; Lewis Phillips G. D, Cancer Res 2008; 68:9280-9290). Incubation times, e.g. cell lysis and incubation with luminescent reagent, were extended to 3 min and 20 min respectively, for optimal luminescent signal. Luminescence was recorded using a plate reader (e.g. MD Spectramax M3 plate reader), and data subsequently analysed using a four parameter non-linear regression model.

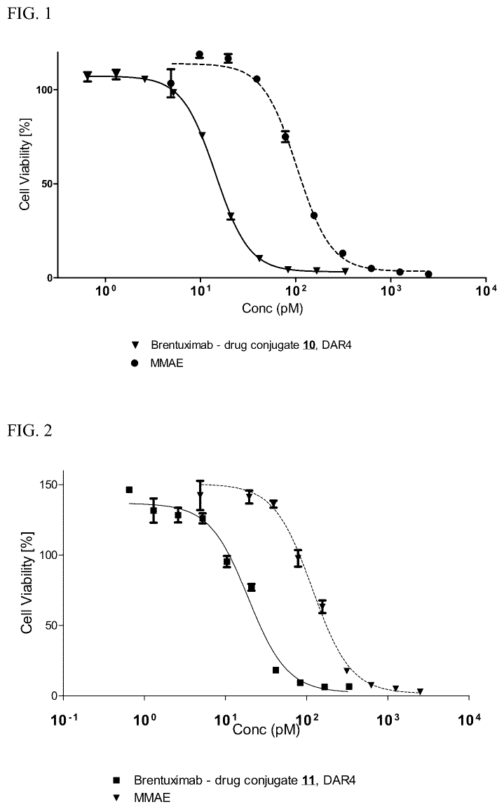

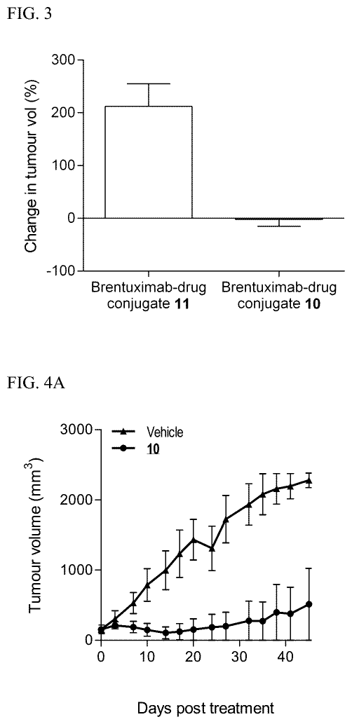

The results are shown in FIGS. 1 and 2, which illustrate cell viability responses to treatment with either antibody conjugates 10, 11 or free drug within Karpas 299 cells. FIG. 1 shows the effect of brentuximab-drug conjugate 10 (solid line) and free drug, MMAE (dashed line), on cell viability of CD30-positive Karpas 299 cell line, while FIG. 2 shows the effect of brentuximab-drug conjugate 11 (solid line) and free drug, MMAE (dashed line), on cell viability of CD30-positive Karpas 299 cell line. Viability is expressed as % of untreated cells. The % viability (Y-axis) is plotted against the logarithm of drug concentration in pM (x-axis) to determine the IC50 values for all conjugates as well as free drug. The IC50 values are shown in Table 2.

TABLE 2

| Sample name | IC50 [pM] | St. Dev |
|---|---|---|
| Brentuximab-drug conjugate 10 | 13.0 | 1.1 |
| Brentuximab-drug conjugate 11 | 19.3 | 2.6 |
| MMAE | 105.3 | 3.4 |

As shown in FIGS. 1 and 2 and Table 2, the antibody-drug conjugates 10 and 11 are active in CD30-positive Karpas 299.

### Example 7: In Vivo Xenograft Study Comparing Brentuximab-Drug Conjugate 10 Prepared from Reagent 2 with the Brentuximab-Drug Conjugate 11 Produced Using the Method Described in WO2014064423

Two purified antibody drug conjugates (ADCs), 10 and 11 each with DAR=4 were produced as described within example 6. Purity following HIC purification was greater than 95% for both conjugates.

Each conjugate was then used in xenograft studies as follows.

Healthy female severe combined immunodeficient (SCID) mice (C.B-17/Icr-Prkdescid, Charles River Laboratories) with an average body weight (BW) of 20.2 g (range=16.5 g to 23.2 g) on Day 1 of the study were used. The animals were maintained in SPF health status according to the FELASA guidelines in housing rooms under controlled environmental conditions. Animal enclosures were designed to provide sterile and adequate space with bedding material, food and water, environmental and social enrichment.

Xenografts were initiated with Karpas 299 T-anaplastic large cell lymphoma (ALCL) cell line by subcutaneous injection in SCID mice. On the day of tumour induction, each test mouse received 107 Karpas 299 cells in 200 μL of RPMI 1640 into the right flank. Tumours were measured in two dimensions using calipers, and volume was calculated using the formula:

$$Tumor Volume ( {mm}^{3} ) = \frac{{w}^{2} \times l}{2}$$

where w=width and l=length, in mm, of the tumour.

Twelve to fourteen days after tumour implantation, designated as Day 1 of the study, the animals were sorted into groups each consisting of five or ten mice with group mean tumour volumes of 111 to 115 mm³ or 148 to 162 mm³. Treatment began on Day 1 in all groups. One treatment group was given intravenous injection (i.v.) on Day 1 with brentuximab-drug conjugate 11 at 1 mg/kg, and another treatment group with brentuximab-drug conjugate 10 at 1 mg/kg. PBS was given to mice in the vehicle-treated control group.

Mice were monitored individually, and each animal was euthanized when its tumour reached the endpoint volume of 2000 mm3. Treatment tolerability was assessed by body weight measurements and frequent observation for clinical signs of treatment-related side effects.

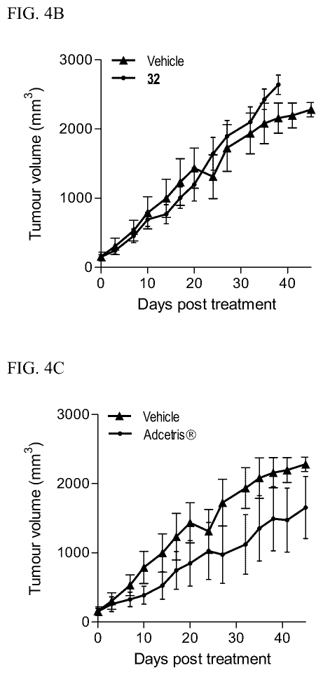

Percentage change in tumour volume was calculated for each mouse at day 7 and expressed as % mean±standard error. All regimens were well tolerated and could be evaluated for efficacy. Percentage tumour volume change after 7 days in the group treated with brentuximab conjugated using brentuximab-drug conjugate 11 was 212±43%, indicating an increase in tumour volume. In contrast, percentage tumour volume change at day 7 in the group treated with brentuximab conjugated using brentuximab-drug conjugate 10 was significantly lower (p=0.0043; student's t test) at −2±6%, indicating a reduction in tumour volume and enhanced anti-tumour effect. The results are shown in FIG. 3, which shows the percentage change in tumour volume for brentuximab-drug conjugates 10 and 11. Conjugates were dosed at 1 mg/kg i.v., and tumour volume measured at day 7 post-injection. Values are expressed as % mean±standard error.

### Example 8: Synthesis of a Disulfide Bridging Reagent 11A Comprising the Cytotoxic Payload 1A

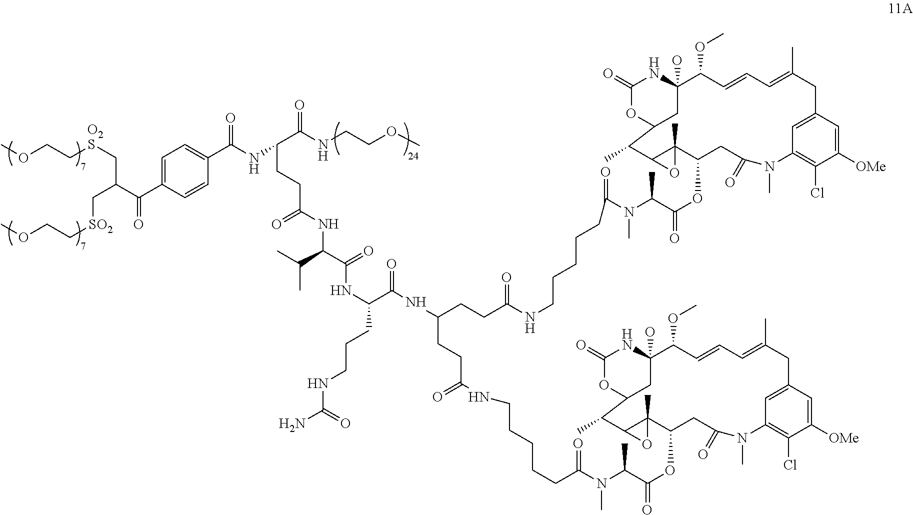

### Synthesis of Cytotoxic Payload 1A.

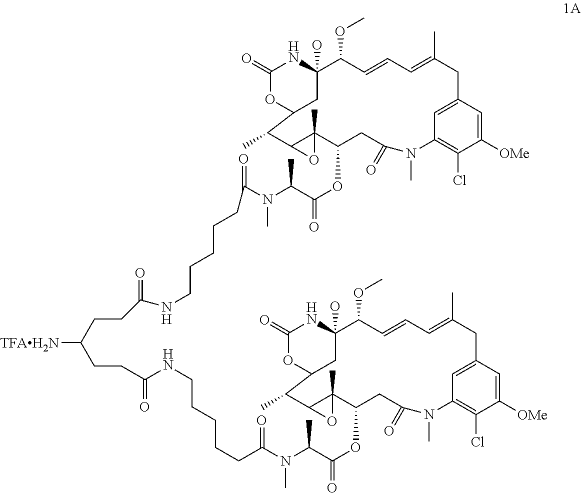

### Step 1: Synthesis of Compound 2A.

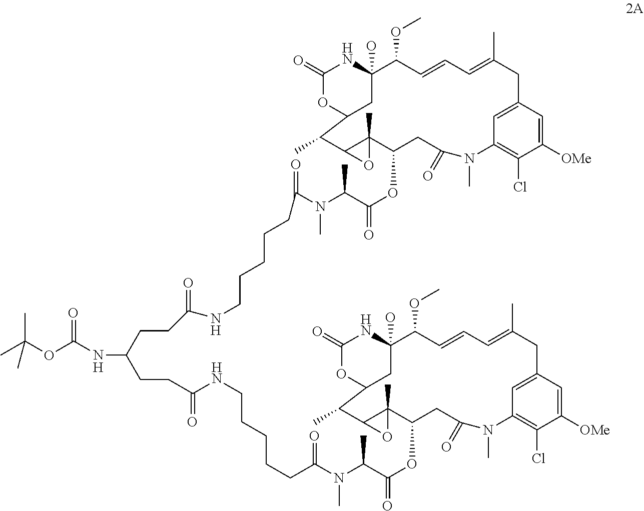 [SMILES: COc1cc2cc(c1Cl)N(C)C(=O)C[C@H](OC(=O)[C@H](C)N(C)C(=O)CCCCCNC(=O)CCC(CCC(=O)NCCCCCC(=O)N(C)[C@@H](C)C(=O)O[C@H]1CC(=O)N(C)c3cc(cc(OC)c3Cl)C/C(C)=C/C=C/[C@@H](OC)[C@@]3(O)CC(OC(=O)N3)[C@@H](C)C3O[C@]31C)NC(=O)OC(C)(C)C)[C@]1(C)OC1[C@H](C)C1C[C@@](O)(NC(=O)O1)[C@H](OC)/C=C/C=C(\C)C2]

To a stirred solution of aminohexanoic maytansine (AHX-DM1). TFA salt (29.4 mg) of formula:

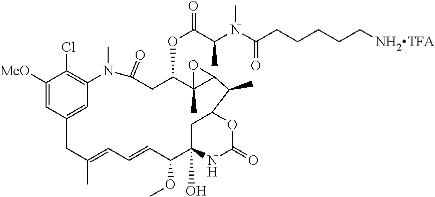

in dimethylformamide (DMF) (400 μL) was added a solution of 4-(N-Boc-amino)-1,6-heptanedioic acid bis-pentafluorophenyl ester (10.2 mg) in DMF (200 μL). The solution was cooled to 0° C. before addition of N,N-diisopropylethylamine (DIPEA) (13.5 μL). The solution was allowed to warm to room temperature and stirred for 18.5 h. The reaction solution was purified by reverse phase C18-column chromatography eluting with buffer A (v/v): water:5% acetonitrile:0.05% trifluoroacetic acid and buffer B (v/v): acetonitrile:0.05% trifluoroacetic acid (100:0 v/v to 0:100 v/v). The solvent was removed by lyophilisation to give 4-(N-boc-amino)-1,6-heptanediamide bis-AHX-DM1 compound 2A (assumed quantitative yield, 29.7 mg) as a white solid m/z [M+2H−2(H₂O)−NHCO]²⁺ 844 (100%), [M+H]⁺ 1767.

### Step 2: Synthesis of Cytotoxic Payload 1A.

Compound 2 (assumed quantitative yield, 29.7 mg) was dissolved in formic acid (700 μL) and the solution stirred at room temperature for 1.5 h. Volatiles were removed in vacuo and the residue converted to the trifluroacetic acid salt by dissolving in a buffer A:buffer B 50:50 v/v % mixture (1.5 mL, buffer A (v/v): water:5% acetonitrile:0.05% trifluoroacetic acid and buffer B (v/v): acetonitrile:0.05% trifluoroacetic acid). The solution was stirred at room temperature for 5 min before the solvent was removed by lyophilisation. The process was repeated to give 4-(amino)-1,6-heptanediamide bis-AHX-DM1 cytotoxic payload 1A as an off-white solid (18.0 mg, 60% over 2 steps) m/z [M+2H−2(H₂O)−NHCO)]²⁺ 794 (100%), [M+H]⁺ 1667.

### Step 3: Synthesis of Compound 8A.

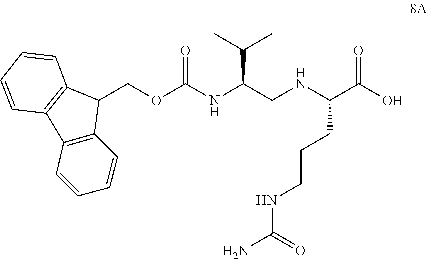 [SMILES: CC(C)[C@@H](CN[C@@H](CCCNC(N)=O)C(=O)O)NC(=O)OCC1c2ccccc2-c2ccccc21]

Compound 8A was synthesised following the procedure described in patent (EP 0 624 377 A2) to give a white solid with spectroscopic data in agreement with that previously reported.

### Step 4: Synthesis of Compound 9A.

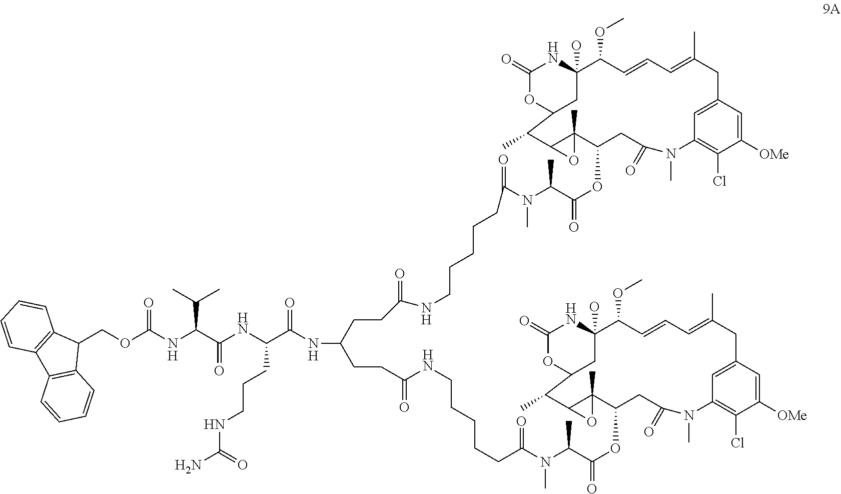 [SMILES: COc1cc2cc(c1Cl)N(C)C(=O)C[C@H](OC(=O)[C@H](C)N(C)C(=O)CCCCCNC(=O)CCC(CCC(=O)NCCCCCC(=O)N(C)[C@@H](C)C(=O)O[C@H]1CC(=O)N(C)c3cc(cc(OC)c3Cl)C/C(C)=C/C=C/[C@@H](OC)[C@@]3(O)CC(OC(=O)N3)[C@@H](C)C3O[C@]31C)NC(=O)[C@H](CCCNC(N)=O)NC(=O)[C@@H](NC(=O)OCC1c3ccccc3-c3ccccc31)C(C)C)[C@]1(C)OC1[C@H](C)C1C[C@@](O)(NC(=O)O1)[C@H](OC)/C=C/C=C(\C)C2]

Stock solutions of compound 8A (20.0 mg) in DMF (500 μL) and HATU (40.0 mg) in DMF (400 μL) were prepared. To a stirred solution of compound 1A (14.0 mg) in DMF (700 μL) was added aliquots of compound 8A stock solution (126.9 μL) and HATU stock solution (77.8 μL).

The reaction solution was cooled to 0° C. before the addition of DIPEA (4.11 μL). The solution was stirred at 0° C. for 50 min before further aliquots of compound 8A stock solution (126.9 μL), HATU stock solution (77.8 μL) and DIPEA (4.11 μL) were added. The solution was stirred for 40 min at 0° C. The reaction solution was purified by reverse phase C18-column chromatography eluting with buffer A (v/v): water:0.05% trifluoroacetic acid and buffer B (v/v): acetonitrile:0.05% trifluoroacetic acid (100:0 v/v to 0:100 v/v). The solvent was removed by lyophilisation to give 4-(Fmoc-val-cit-amido)-1,6-heptanediamide bis-AHX-DM1 compound 9A (assumed quantitative yield, 16.9 mg) as an off-white solid m/z [M+2H−2(H₂O)]²⁺ 1055 (100%).

### Step 5: Synthesis of Compound 10A.

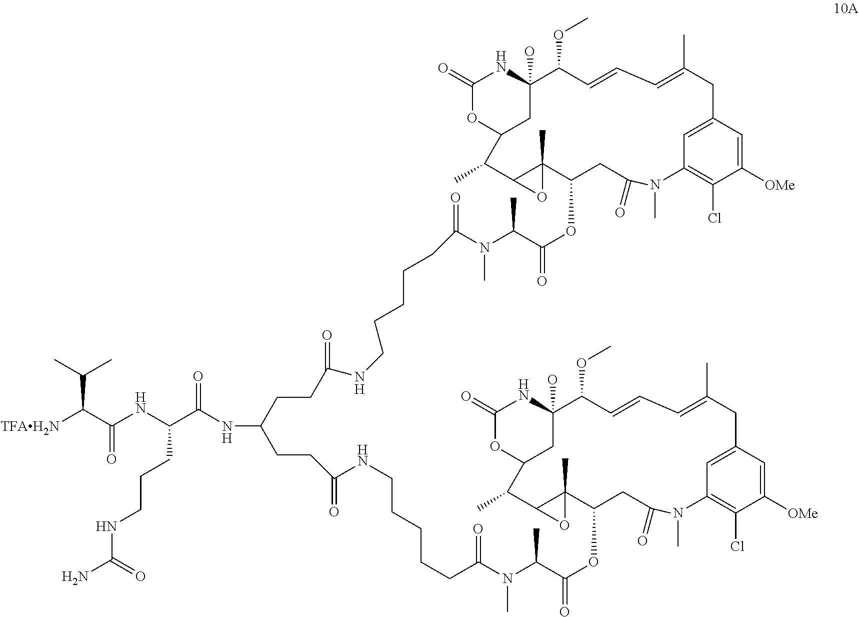

To a stirred solution of compound 9A (assumed quantitative yield, 16.9 mg) in DMF (500 μL) was added piperidine (3.04 μL). The reaction solution was stirred at room temperature for 1.5 h before purification by reverse phase C18-column chromatography eluting with buffer A (v/v): water:0.05% trifluoroacetic acid and buffer B (v/v): acetonitrile:0.05% trifluoroacetic acid (100:0 v/v to 0:100 v/v). The solvent was removed by lyophilisation to give 4-(val-cit-amido)-1,6-heptanediamide bis-AHX-DM1 compound 10A as an off-white solid (8.8 mg, 55% over 2 steps) m/z [M+2H]²⁺ 962 (100%).

### Step 6: Synthesis of Compound 12A.

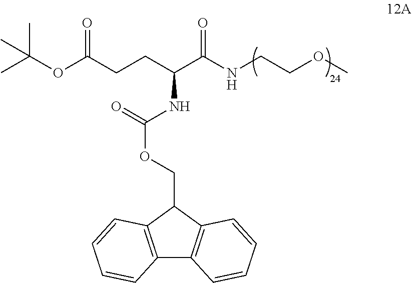

A solution of Fmoc-L-Glu-(OtBu)-OH (36 mg) in DMF (2 mL) under an argon atmosphere was cooled to 0° C. and (benzotriazol-1-yloxy)tris-(dimethylamino) phosphonium hexafluorophosphate (BOP) (41 mg) was added followed by NH₂-PEG(24u)-OMe (100 mg) and DIPEA (19 μL). The solution was allowed to warm to room temperature and after 22 h the volatiles were removed in vacuo. The resulting residue was dissolved in dichloromethane (1 mL) and purified by normal phase column chromatography eluting with dichloromethane:methanol (100:0 v/v to 80:20 v/v). The organic solvent was removed in vacuo to give the Fmoc-Glu-(OtBu)-NH-PEG(24u)-OMe compound 12A as a colourless oil (84 mg, 67%) m/z [M+H]⁺ (1097, 10%), [M+2H]²⁺ (1035,100%).

### Step 7: Synthesis of Compound 13A.

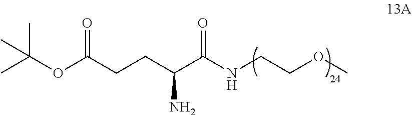

To a solution of compound 12A (74 mg) in DMF (2 mL) under an argon atmosphere was added piperidine (49 μL) and the resulting solution was stirred at room temperature. After 22 h, the volatiles were removed in vacuo and the resulting residue was triturated with hexane (3×0.7 mL). The organic solvent was decanted each time and resulting residue dried in vacuo to give the Glu-(OtBu)-NH-PEG(24u)-OMe compound 13A as a solid (61 mg, 97%) m/z [M+H]⁺ (1097, 10%), [M+2H]²⁺ (1035,100%).

### Step 8: Synthesis of Compound 4A

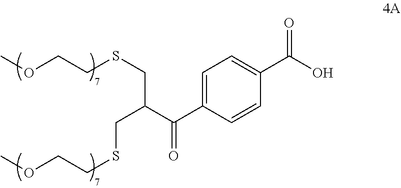

To a stirred solution of 4-[2,2-bis[(p-tolylsulfonyl)-methyl]acetyl]benzoic acid (1.50 g, Nature Protocols, 2006, 1(54), 2241-2252) in DMF (70 mL) was added alpha-methoxy-omega-mercapto hepta(ethylene glycol) (3.20 g) and triethylamine (2.50 mL). The resulting reaction mixture was stirred under an inert nitrogen atmosphere at room temperature. After 19 h, volatiles were removed in vacuo. The resulting residue was dissolved in water (2.4 mL), and purified by reverse phase C18-column chromatography eluting with buffer A (v/v): water:5% acetonitrile:0.05% trifluoroacetic acid and buffer B (v/v): acetonitrile:0.05% trifluoroacetic acid (100:0 v/v to 0:100 v/v). The organic solvent was removed in vacuo and the aqueous solvent removed by lyophilisation to give 4-[2,2-bis[alpha-methoxy-omega-thio-hepta(ethylene glycol)]acetyl]-benzoic acid compound 4A as a thick clear colourless oil (1.77 g, 66%) m/z [M+H]⁺ 901.

### Step 9: Synthesis of Compound 5A.

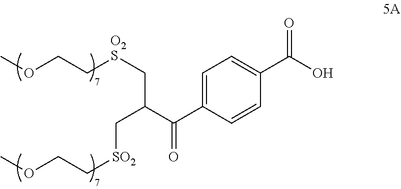

To a stirred solution of 4A (1.32 g) in methanol:water (18 mL, 9:1 v/v) at room temperature was added Oxone© (2.70 g). After 2.5 h, the volatiles were removed in vacuo and water was azeotropically removed with acetonitrile (2×15 mL). The resulting residue was dissolved in dichloromethane (3×10 mL), filtered through a column of magnesium sulphate and washed with dichloromethane (2×7 mL). The eluent and washings were combined and the volatiles were removed in vacuo to give a thick clear pale yellow oil (1.29 g, 92%). A portion of the residue (700 mg) was dissolved in water:acetonitrile (1.50 mL, 3:1 v/v), and purified by reverse phase C18-column chromatography eluting with buffer A (v/v): water:5% acetonitrile:0.05% trifluoroacetic acid and buffer B (v/v): acetonitrile:0.05% trifluoroacetic acid (100:0 v/v to 0:100 v/v). The organic solvent was removed in vacuo and the aqueous solvent removed by lyophilisation to give 4-[2,2-bis[alpha-methoxy-omega-sulfonyl hepta(ethylene glycol)]acetyl]benzoic acid reagent 5A as a thick clear colourless oil (524 mg, 68%) m/z [M+H]⁺ 965.

### Step 10: Synthesis of Compound 14A.

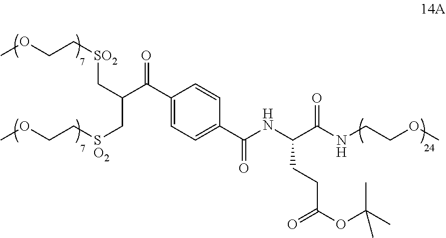

A solution of compound 5A (26.6 mg) in DMF (550 μL) was cooled to 0° C. under an argon atmosphere when HATU (10.5 mg) was added and the solution was stirred for 0.5 h at 0° C. To this was added a solution of 13A (32 mg) in DMF (550 μL) and the resulting solution was stirred for 5 min at 0° C. before addition of NMM (2.9 μL) and HATU (10.5 mg). The reaction solution was allowed to stir at 0° C. for 2 h before being warmed to room temperature and stirred for a further 3.5 h. After this time the volatiles were removed in vacuo, the resulting residue dissolved in water and acetonitrile (v/v; 1/1, 1.2 mL), and purified by reverse phase C18-column chromatography eluting with buffer A (v/v): water:5% acetonitrile:0.1% formic acid and buffer B (v/v): acetonitrile:0.1% formic acid (100:0 v/v to 0:100 v/v). The organic solvent was removed in vacuo and the aqueous solvent removed by lyophilisation to give the bis-mPEG(7u)sulfone-propanoyl-benzamide-Glu-(OtBu)-NH-PEG(24u)-OMe compound 14A as a colourless oil (30.5 mg, 55%) m/z [M+Na]⁺ (2243, 50%), [M+H]⁺ (2221, 40%), [M+Na+2H]³⁺ (747,100%).

### Step 11: Synthesis of Compound 15A.

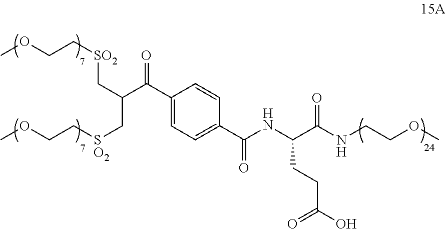

A solution of compound 14A (30 mg) in dichloromethane (2 mL) under an argon atmosphere was cooled to 0° C. after which trifluoroacetic acid (500 μL) was added and the resulting solution stirred for 1.5 h. The reaction mixture was allowed to warm to room temperature and stirred for a further 1 h, after which time the volatiles were removed in vacuo. The resulting residue was dissolved in water and acetonitrile (v/v; 1/1, 0.6 mL), and purified by reverse phase C18-column chromatography eluting with buffer A (v/v): water:5% acetonitrile:0.05% trifluoroacetic acid and buffer B (v/v): acetonitrile:0.05% trifluoroacetic acid (100:0 v/v to 0:100 v/v). The organic solvent was removed in vacuo and the aqueous solvent removed by lyophilisation to give the bis-mPEG(7u)sulfone-propanoyl-benzamide-Glu-NH-PEG(24u)-OMe compound 15A as a colourless oil (20 mg, 68%) m/z [M+H]⁺ (2165, 55%), [M+2H]²⁺ (1083, 60%), [M+2H+Na]³⁺ (729, 100%).

### Step 12: Synthesis of Reagent 11A.

Stock solutions of HATU (10 mg) in DMF (200 μL) and NMM (5.83 μL) in DMF (94.2 μL) were prepared. Compound 15A (5.4 mg) was dissolved in a solution of compound 10A (3.6 mg) in DMF (153.7 μL) with stirring. To the stirred solution was added an aliquot of HATU stock solution (40 μL). The solution was cooled to 0° C. before an aliquot of NMM stock solution (10 μL) was added. After 50 min, further aliquots of HATU stock solution (6.67 μL) and NMM stock solution (1.67 μL) were added. The reaction solution was stirred at 0° C. for a further 30 min and purified directly by reverse phase C18-column chromatography eluting with buffer A (v/v): water:0.05% trifluoroacetic acid and buffer B (v/v): acetonitrile:0.05% trifluoroacetic acid (100:0 v/v to 0:100 v/v). The solvent was removed by lyophilisation to give reagent 11A as an off-white solid (3.8 mg, 53%) m/z [M+4H−(H₂O)−NHCO]⁴⁺ 1003 (100%), [M+3H−2(H₂O)−NHCO]³⁺ 1331, [M+2H−2(H₂O)]²⁺ 2017.

### Example 9: Synthesis of a Disulfide Bridging Reagent 16A Comprising the Cytotoxic Payload 1A

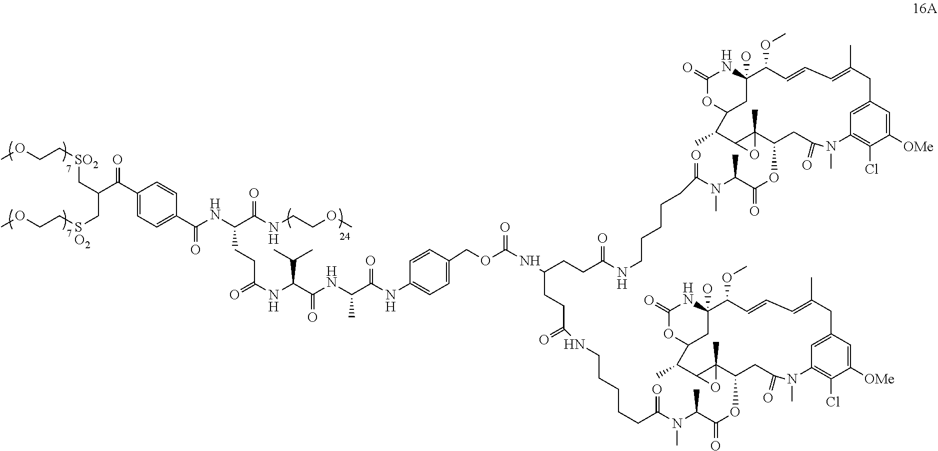

### Step 1: Synthesis of Compound 17A.

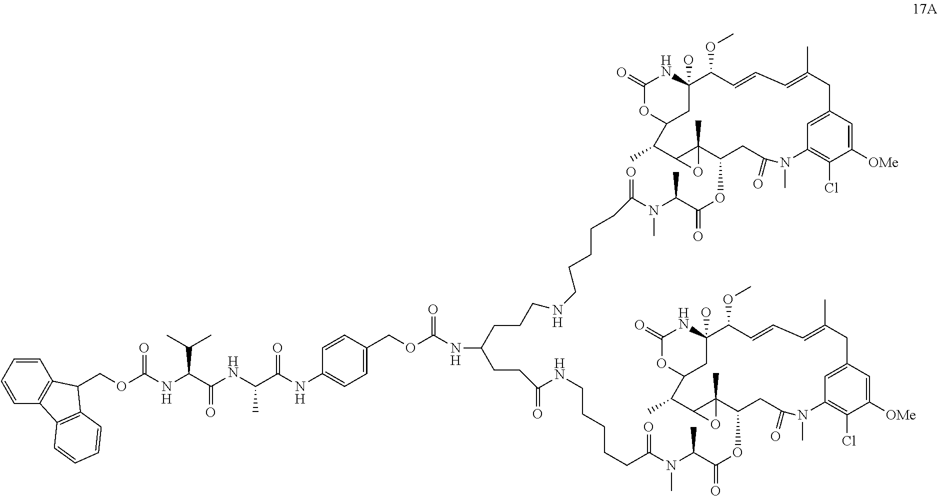 [SMILES: COc1cc2cc(c1Cl)N(C)C(=O)C[C@H](OC(=O)[C@H](C)N(C)C(=O)CCCCCNCCCC(CCC(=O)NCCCCCC(=O)N(C)[C@@H](C)C(=O)O[C@H]1CC(=O)N(C)c3cc(cc(OC)c3Cl)C/C(C)=C/C=C/[C@@H](OC)[C@@]3(O)CC(OC(=O)N3)[C@@H](C)C3O[C@]31C)NC(=O)OCc1ccc(NC(=O)[C@H](C)NC(=O)[C@@H](NC(=O)OCC3c4ccccc4-c4ccccc43)C(C)C)cc1)[C@]1(C)OC1[C@H](C)C1C[C@@](O)(NC(=O)O1)[C@H](OC)/C=C/C=C(\C)C2]

A stock solution of hydroxybenzotriazole (HOBt, 6.6 mg) in DMF (200 μL) was prepared. To a stirred solution of cytotoxic payload 1A (10 mg) in DMF (500 μL) was added Fmoc-val-ala-PAB-PNP (3.7 mg) and an aliquot of HOBt stock solution (2 μL). The reaction solution was cooled to 0° C. before DIPEA (2.14 μL) was added. The reaction solution was then stirred at room temperature for 18 h before purification by reverse phase C18-column chromatography, eluting with buffer A (v/v): water:0.05% trifluoroacetic acid and buffer B (v/v): acetonitrile:0.05% trifluoroacetic acid (100:0 v/v to 0:100 v/v). The solvent was removed by lyophilisation to give Fmoc-val-ala-PAB-amido-1,6-heptanediamide bis-AHX-DM1 reagent 17A.

### Step 2: Synthesis of Compound 18A.

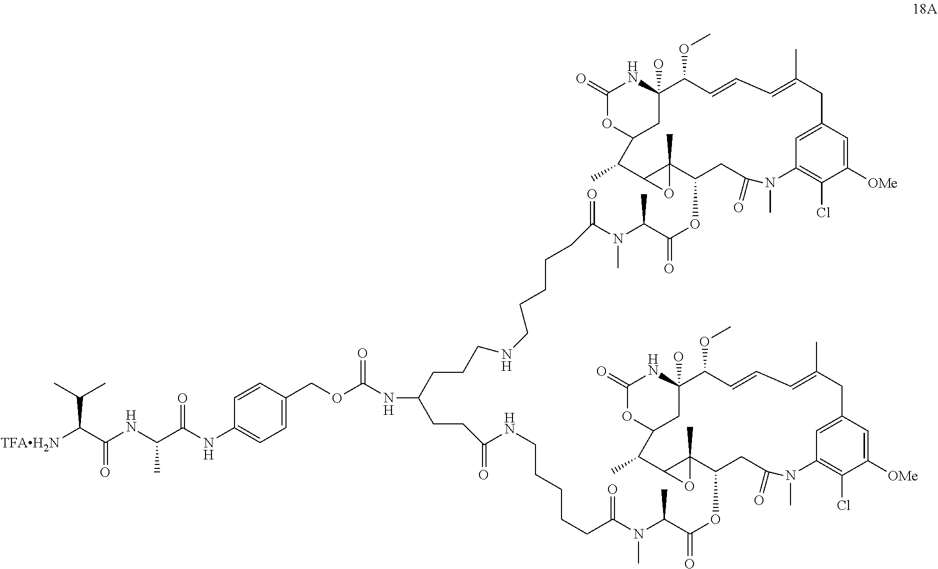

The bis-maytansinoid compound amine-val-ala-PAB-amido-1,6-heptanediamide bis-AHX-DM1 18A was synthesised in an analogous way to that described for compound 10A, using compound 17A instead of compound 9A.

### Step 3: Synthesis of Reagent 16A.

The bis-maytansinoid reagent bis-mPEG(7u)sulfone-propanoyl-benzamide-Glu-[NH-PEG(24u)-OMe]-[val-ala-PAB-amido-1,6-heptanediamide bis-AHX-DM1] 16A was synthesised in an analogous way to that described for reagent 11A, using compound 18A instead of compound 10A.

### Example 10: Synthesis of Conjugation Reagent 13 Comprising an Auristatin Cytotoxic Payload

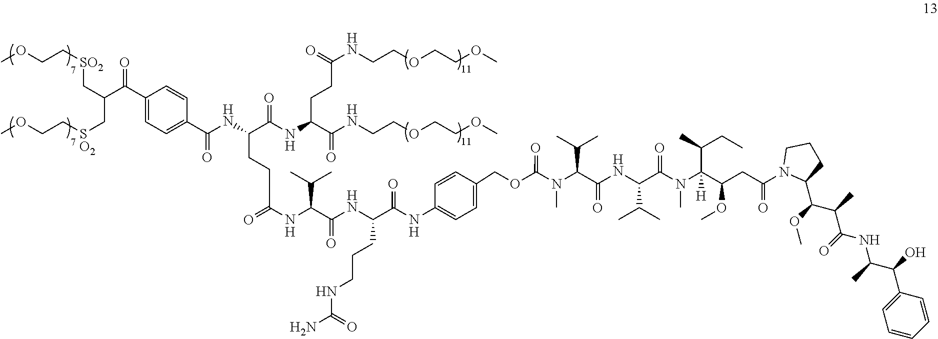

### Step 1: Synthesis of Compound 14.

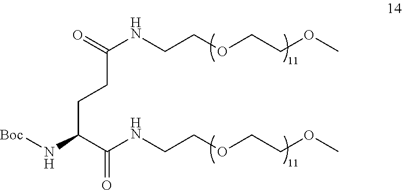

Boc-L-Glu (134.9 mg) and (benzotriazol-1-yloxy)tris-(dimethylamino) phosphonium hexafluorophosphate (BOP) (724 mg) were dissolved in anhydrous DMF (4 mL) and were stirred at 0° C. under a nitrogen atmosphere for 1.25 h. This solution was then added to a solution of H₂N-PEG(12u)-Me (685 mg) and NMM (179.8 μL) in DMF (3 mL). The solution was then stirred under N₂ for 4 h. The solution was then stirred 0-4° C. under a nitrogen atmosphere for 4.5 h. Further BOP (241 mg) and NMM (60 μL) were added, reaction mixture left for 24 h at 4° C. The volatiles were removed in vacuo and the resulting residue was purified by reverse phase C18-flash chromatography eluting with eluting with buffer A (v/v): water:5% acetonitrile:0.1% formic acid and buffer B (v/v): acetonitrile:0.1% formic acid (100:0 v/v to 65:35 v/v). The organic solvent was removed in vacuo and the aqueous solvent was removed by lyophilisation. The material was repurified by normal phase flash chromatography eluting with ethyl acetate:methanol (100:0 v/v to 0:100 v/v). The organic solvent was removed in vacuo and the aqueous solvent was removed by lyophilisation to give Boc-Glu-[PEG(12u)-Me]2 compound 14 as a colourless oil (450 mg). m/z [M+H]⁺ (1331, 100%), [M+2H]²⁺ (665, 100%).

### Step 2: Synthesis of Compound 15.

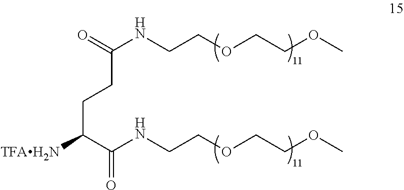

Compound 14 (450 mg) was dissolved in DCM (25 mL) to which was added TFA (2.5 mL). The solution stirred at room temperature for 5 h. After which the volatiles were removed in vacuo. The resulting residue was purified by reverse phase C18-flash chromatography eluting with buffer A (v/v): water:5% acetonitrile:0.05% trifluoroacetic acid and buffer B (v/v): acetonitrile:0.05% trifluoroacetic acid (100:0 v/v to 60:40 v/v). The organic solvent was removed in vacuo and the aqueous solvent was removed by lyophilisation to give Glu-[HN-PEG(12u)-Me]2 TFA compound 15 as a clear colourless gum (320 mg) m/z [M+Na]¹⁺ (1253.0, 10%) [M+H]²⁺ (616.8, 100%)

### Step 3: Synthesis of Compound 16

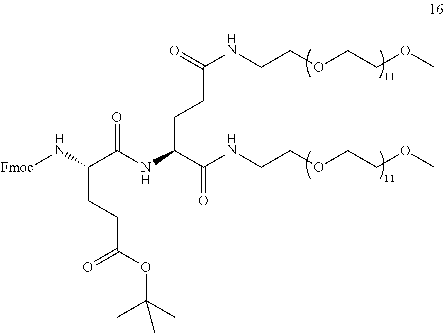

To a stirred solution of Fmoc-L-Glu-(OtBu)-OH (36.6 mg) in anhydrous DMF (2 mL) was added HATU (37.30 mg). The reaction mixture was stirred at 0° C. under a nitrogen atmosphere for 1 h and then added to a solution of compound 15 (103.5 mg) and NMM (19.2 μL) in DMF (1 mL). Additional DMF (1 mL) was added. The stirred reaction was left to warm to room temperature over 5 h. The volatiles were removed in vacuo. The resulting pale yellow oil was purified by reverse phase C18-flash chromatography eluting with buffer A (v/v): water:5% acetonitrile:0.1% formic acid and buffer B (v/v): acetonitrile:0.1% formic acid (100:0 v/v to 50:50 v/v). The organic solvent was removed in vacuo and the aqueous solvent was removed by lyophilisation to give Fmoc-L-Glu-(O^{t}Bu)-Glu-[HN-PEG(12u)-Me]2 compound 16 (173 mg) as a white paste. m/z [M+1]⁺ (1638, 100%) & [M+Na]⁺ (1660, 57%).

### Step 4: Synthesis of Compound 17

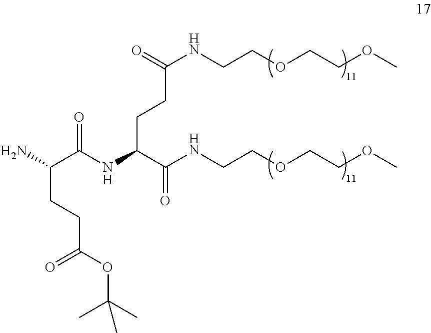

To a stirred solution of compound 16 (173 mg) in anhydrous DMF (3.2 mL) was added piperidine (104.4 μL). The solution was stirred at room temperature under argon for 1.5 h. The volatiles were removed in vacuo and the residue triturated repeatedly with hexane. The product was dried in vacuo to give L-Glu-(O^{t}Bu)-L-Glu-[HN-PEG(12u)-Me]2 compound 17 (152 mg) as a clear colourless oil. m/z [M+H]¹⁺ (1416.7, 85%), [M+2H]²⁺ (708.5, 100%), [M+Na]¹⁺ (1438.7, 30%)

### Step 5: Synthesis of Compound 18

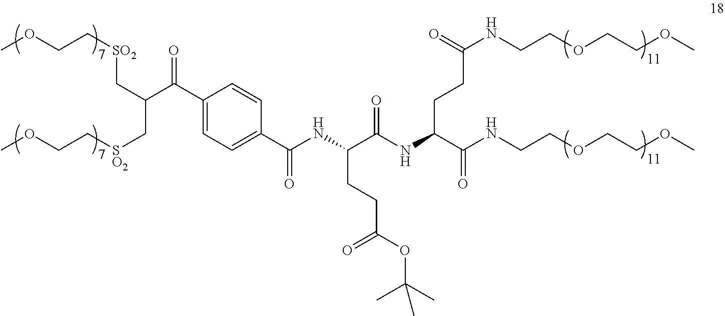

To a stirred solution of 4-[2,2-bis[alpha-methoxy-omega-sulfonyl hepta(ethylene glycol)]acetyl]benzoic acid (114 mg) in anhydrous DMF (3 mL) was added HATU (51.4 mg). Reaction mixture was stirred at 0° C. for 0.5 h then added to a solution of L-Glu(O^{t}Bu)-Glu-[HN-PEG(12u)-OMe]2. (152.0 mg) in DMF (2 mL) and washed in with further DMF (1 mL), followed by NMM (14.8 μL). The reaction mixture was stirred at 0-15° C. for 3.5 h after which the volatiles were removed in vacuo. The resulting residue was purified by reverse phase C18-flash chromatography eluting with buffer A (v/v): water:5% acetonitrile:0.1% formic acid and buffer B (v/v): acetonitrile:0.1% formic acid (100:0 v/v to 55:45 v/v). The organic solvent was removed in vacuo and the aqueous solvent was removed by lyophilisation to give Bis-mPEG(7u)sulfone-propanoyl-benzamide-L-Glu-(O^{t}Bu)-Glu-[HN-PEG(12u)-Me]2 compound 18 (160.6 mg) as a clear colourless oil. m/z [M+H]¹⁺ (2366.7, 100%), [M+2H]²⁺ (1184.0, 80%) [M+H₂O]³⁺ (795.5, 100%).

### Step 6: Synthesis of Compound 19

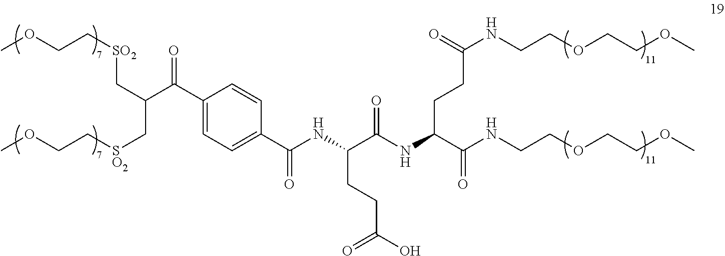

To the stirred solution of compound 18 (58 mg) in anhydrous DCM (6 mL) was added TFA (6.0 mL). Reaction mixture was stirred at room temperature for 2 h. after which the volatiles were removed in vacuo, dissolved in water (25 mL) and lyophilised to give Bis-mPEG(7u)sulfone-propanoyl-benzamide-L-Glu-(OH)-Glu-[HN-PEG(12u)-OMe]2 compound 19 (160.6 mg) as a clear colourless oil. m/z [M+H]¹⁺ (2306.8, 90%), [M+2H]²⁺ (1153.0, 100%).

### Step 7: Synthesis of Reagent 13

Reagent 13 was synthesised in analogous way to reagent 8 of Example 3 from compound 7 and val-cit-PAB-MMAE TFA salt. Bis-mPEG(7u)sulfone-propanoyl-benzamide-L-Glu-(val-cit-PAB-MMAE)-Glu-[HN-PEG(12u)-Me]₂ 13 was isolated as a colourless oil (69%). m/z [M+H]¹⁺ (3410.4, 90%), [M+2H]²⁺ (1706.2, 60%), [M+3H]³⁺ (1137.2, 85%), [M+4H]⁴⁺ (852.8, 70%).

### Example 11: Synthesis of Conjugation Reagent 20 Comprising an Auristatin Cytotoxic Payload

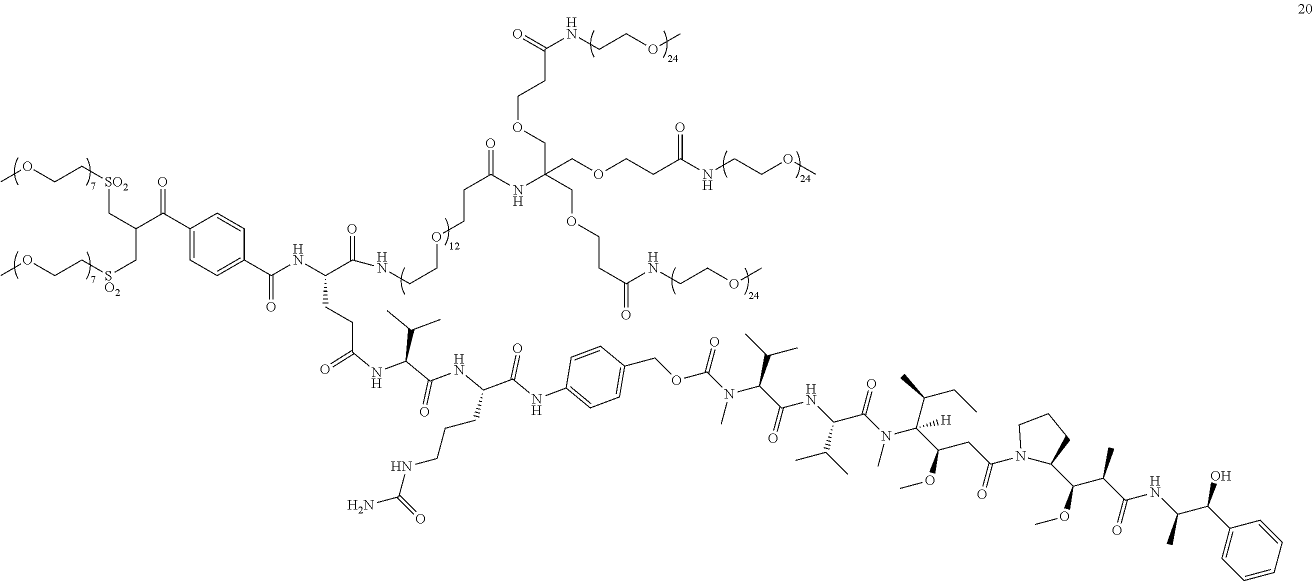

Reagent 20 was synthesised in analogous way to reagent 8 of Example 3 using compound 20B instead of compound 7 and val-cit-PAB-MMAE TFA salt.

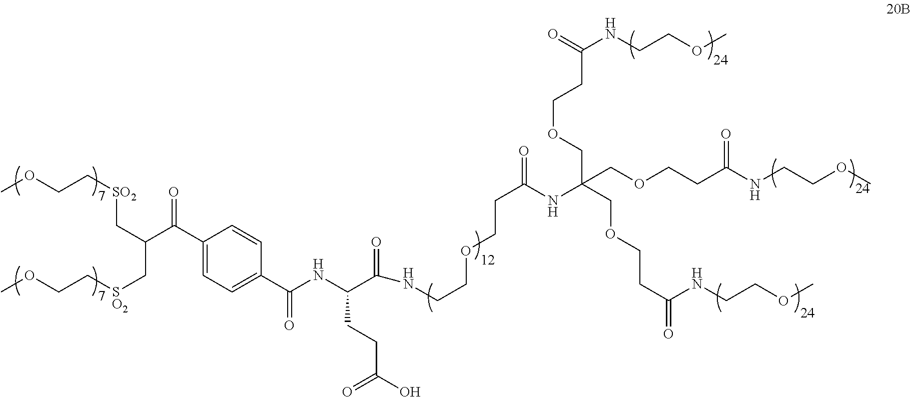

Compound 20B was made in an analogous way to compound 7 in Example 3, using H₂N-PEG(12u)-tri(m-dPEG(24u) instead of H₂N-PEG(24u). Bis-mPEG(7u)sulfone-propanoyl-benzamide-L-Glu-[val-cit-PAB-MMAE]-[PEG(12u)-tri(m-dPEG(24u))] 20 was isolated as a colourless oil. m/z [M+2H]²⁺ (3166, 20%), [M+3H]³⁺ (2111, 50%), [M+4H]⁴⁺ (1583, 100%).

### Example 12: Synthesis of Conjugation Reagent 21 Comprising an Auristatin Cytotoxic Payload

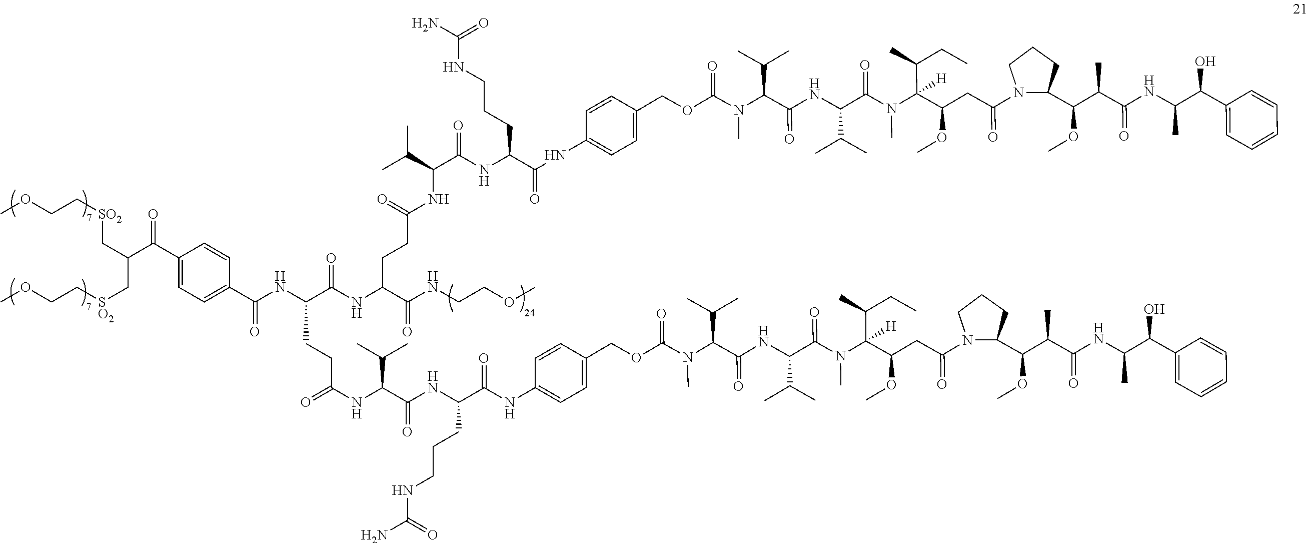

### Step 1: Synthesis of Compound 22.

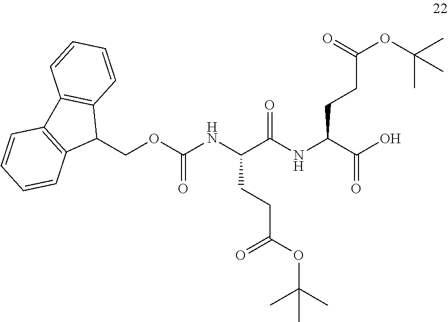 [SMILES: CC(C)(C)OC(=O)CC[C@H](NC(=O)[C@H](CCC(=O)OC(C)(C)C)NC(=O)OCC1c2ccccc2-c2ccccc21)C(=O)O]

To a stirred solution of Fmoc-L-Glu-(O^{t}Bu)-OH (2000 mg) in anhydrous DMF (18 mL) was added HOBt (666 mg) and DIC (768 μL). The reaction mixture was stirred at 0° C. for 10 min and then 2.5 h at room temperature. H-L-Glu-(O^{t}Bu)-OH (1194 mg) and DIPEA (2464 μL) were added and the reaction mixture was stirred for 18 h at room temperature. The reaction mixture was diluted with water (100 mL) and acidified to pH 2.0 by adding diluted HCl. The aqueous layer was extracted with EtOAc (3×100 mL), and the organic phases combined and washed with water (2×50 mL) and saturated brine solution (1×50 mL). The EtOAc layer was dried over Na₂SO₄ for 2 h and then concentrated on a rotary evaporator. The product was isolated by reverse phase C18-flash chromatography eluting with buffer A (v/v): water: 5% acetonitrile: 0.1% formic acid and buffer B (v/v): acetonitrile: 0.1% formic acid (100:0 v/v to 80:20 v/v). The organic solvent was removed in vacuo and the aqueous solvent was removed by lyophilisation to give compound Fmoc-L-Glu-(OtBu)-L-Glu-(OtBu)-OH 22 (875 mg) as a white solid. m/z [M+H]¹⁺ (610.8, 85%), [M+Na]¹⁺ (633.1, 55%), [2M+Na]⁺ (1243.2, 55%).

### Step 2: Synthesis of Compound 23.

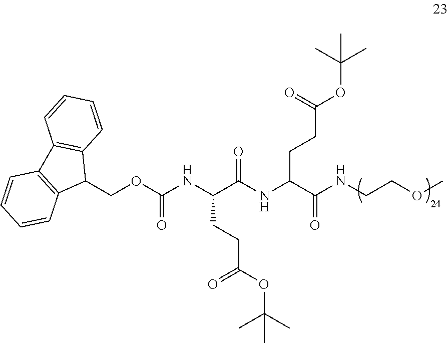

To a stirred solution of Fmoc-L-Glu-(OtBu)-L-Glu-(OtBu)-OH (510 mg) and NH₂-PEG(24u)-OMe (1000 mg) in anhydrous DMF (5 mL) was added and N,N-diisopropylethylamine (43.8 μL) and HATU (47.6 mg). The reaction mixture was stirred at 0° C. for 10 min and then 16 h at room temperature. The solution was concentrated in vacuo to 2 mL and the residue was purified by reverse phase C18-flash chromatography eluting with buffer A (v/v): water:5% acetonitrile:0.1% formic acid and buffer B (v/v): acetonitrile:0.1% formic acid (100:0 v/v to 83:17 v/v). The organic solvent was removed in vacuo and the aqueous solvent was removed by lyophilisation to give Fmoc-Glu-(OtBu)-Glu-(OtBu)-PEG(24u)-OMe compound 23 644 mg) as a white paste. m/z [M+H]¹⁺ (1681.0, 40%), [M+Na]¹⁺ (1704.0, 30%) and [M+2H]²⁺ (841.4, 55%).

### Step 3: Synthesis of Compound 24.

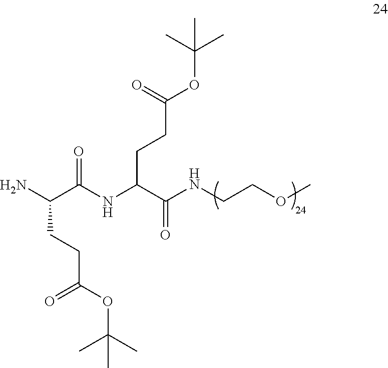

To a stirred solution of Fmoc-Glu-(OtBu)-Glu-(OtBu)-PEG(24u)-OMe (193 mg) in anhydrous DMF (900 μL) was added piperidine (34 μL) and the reaction mixture was stirred 1 h at room temperature. The solution was concentrated in vacuo to dryness and the residue triturated with Et₂O (2×2.5 mL). The product was dried in vacuo to give H-L-Glu-(OtBu)-Glu-(OtBu)-PEG(24u)-OMe compound 24 (166 mg) as an off-white solid.

### Step 4: Synthesis of Compound 25.

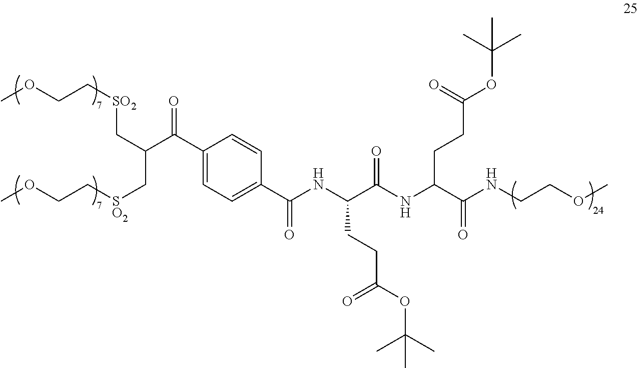

Reagent 25 was synthesised in analogous way to reagent 18 of Example 8 from compound 24 and 4-[2,2-bis[alpha-methoxy-omega-sulfonyl hepta(ethylene glycol)]acetyl]benzoic acid. Bis-mPEG(7u)sulfone-propanoyl-benzamide-Glu-(OtBu)-Glu-(OtBu)-PEG(24u)-OMe 25 was isolated as a colourless oil. m/z [M+H]¹⁺ (2407.2, 25%), [M+Na]¹⁺ (2429.4, 70%).

### Step 5: Synthesis of Compound 26.

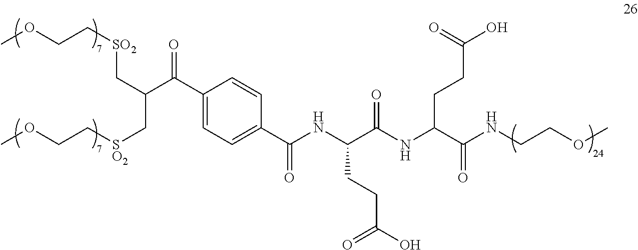

Reagent 26 was synthesised in analogous way to reagent 19 of Example 8 from compound 25. Bis-mPEG(7u)sulfone-propanoyl-benzamide-Glu-(OH)-Glu-(OH)-PEG(24u)-OMe 26 was isolated as a colourless oil. m/z [M+H]¹⁺ (2294.2, 20%), [M+Na]¹⁺ (2317.4, 10%) and [M+2Na]²⁺ (1217.4, 100%).

### Step 6: Synthesis of Reagent 21.

To a stirred solution of compound 26 (28.1 mg), val-cit-PAB-MMAE TFA salt (30.6 mg) and HATU (13.9 mg) in anhydrous DMF (1.5 mL) was added N-methylmorpholine (6.7 μL) and the reaction mixture was stirred at 0° C. for 5 h. The solution was diluted with water (1 mL) and purified by reverse phase C18-flash chromatography eluting with buffer A (v/v): water:5% acetonitrile:0.1% TFA and buffer B (v/v): acetonitrile:0.1% TFA (100:0 v/v to 60:40 v/v). The organic solvent was removed in vacuo and the aqueous solvent was removed by lyophilisation to give bis-mPEG(7u)sulfone-propanoyl-benzamide-bis-[Glu-(val-cit-PAB-MMAE)]-PEG(24u)-OMe compound 21 (36.1 mg) as a white solid. m/z [M+2H]²⁺ (2252.7, 20%), [M+3H]3+(1501.6.7, 40%) and [M+4H]4⁺ (1126.6, 100%).

### Example 13. Analysis of Antibody Drug Conjugates (ADCs) by In Vitro Cell Viability Assay

The in vitro efficacy of the antibody conjugates and free payloads prepared in Example 5 were determined by measuring the inhibitory effect on cell growth of target over-expressing cancer cell lines.

Loss of tumour cell viability following treatment with ADCs or free payloads in vitro can be measured by growing cell lines in the presence of increasing concentrations of compounds and quantifying the loss of proliferation or metabolic activity using Cell-Titer Glo® Luminescent reagent (Promega). The protocol describes cell seeding, drug treatment and determination of the cell viability in reference to untreated cells based on ATP synthesis, which is directly correlated to the number of cells present in the well.

The characteristics of the cell line as well as the seeding density for the assay are described in Table 3 below.

Adherent JIMT-1 cells were detached with TrypLE and resuspended in complete medium. Cells were counted using disposable Neubauer counting chambers and cell density adjusted as detailed in Table 3 below. Cells were seeded (adherent cells at 100 μL/well and Karpas-299 at 50 μL/well) into Tissue Culture treated opaque-walled 96-well white plates and incubated for 24 hrs at 37° C. and 5% CO₂.

TABLE 3

| Cell | | | Seeding |
|---|---|---|---|
| line | Target | Growth Medium | density |
| JIMT-1 | Her2^{low} | DMEM medium (Life Technologies ®), | 0.3 × 10⁴ |
| | | 10% fetal bovine serum, 100 U/mL | cells per well |
| | | Penicillin and 100 μg/mL Streptomycin | |

Eight point serial dilutions of compounds were prepared in the relevant culture medium. The titration range was adjusted for each compound/cell line combination. In the case of adherent cells, the medium from the plate containing the cells was removed and replaced by 100 μL/well of the 1× serially diluted compounds.

The cell viability assay was carried out using the Cell-Titer Glo® Luminescent reagent (Promega), as described by the manufacturer.

Luminescence was recorded using a Molecular Devices SpectramaxM3 plate reader and data subsequently analysed using GraphPad Prism four parameter non-linear regression model.

Viability was expressed as % of untreated cells and calculated using the following formula:

$$\% Viability = 100 \times \frac{{Luminescence}_{Sample} - {Luminescence}_{No cell Control}}{{Luminescence}_{Untreated} - {Luminescence}_{No cell Control}}$$

The % viability was plotted against the logarithm of drug concentration to extrapolate the IC₅₀ values for all conjugates.

### Example 14: Karpas-299 Mouse Xenograft Studies Comparing Brentuximab-Drug Conjugate 10 with Brentuximab-Drug Conjugate 46 (Comparative), Trastuzumab-Drug Conjugate 32 (Negative Control), and Adcetris® (Comparative)

In this example, Trastuzumab-drug conjugate 32 was used as a negative control. Trastuzumab binds the target HER-2 which is not present, or present at very low levels, on Karpas-299 cells. Thus Trastuzumab-drug conjugate 32 is not specifically targeted at these cells and should only have a non-specific cytotoxic effect. Conversely, Brentuximab-drug conjugate 10 recognises the cell surface marker CD30, which is expressed upon Karpas-299 cells, targeting the ADC specifically to these cells resulting in a specific cytotoxic effect.

Brentuximab drug conjugates 10 and 46 were prepared from conjugation reagents 2 and 45 respectively by the method described in Example 5. Trastuzumab drug conjugate 32 was prepared from conjugation reagent 1 by the method described in Example 5.

Healthy female CB17 SCID mice (CB17/lcr-Prkdcscid/Crl) with an average body weight of 19.7 g were used for cell inoculation. 24 to 72 hours prior to tumour cell injection, the mice were γ-irradiated (1.44 Gy, ⁶⁰Co). The animals were maintained in SPF health status according to the FELASA guidelines in housing rooms under controlled environmental conditions.

Tumours were induced by subcutaneous injection of 10⁷ Karpas-299 cells (T-anaplastic large cell lymphoma, ALCL) in 200 μL of RPMI 1640 into the right flank. Tumours were measured twice a week with calipers, and the volume was estimated using the formula:

$$Tumor Volume ( {mm}^{3} ) = \frac{{width}^{2} \times length}{2}$$

Fourteen days after tumour implantation, the animals were randomised into groups of five mice using Vivo Manager® software (152.9 mm³ mean tumor volume) and treatment was initiated (Day 0). All test substances were injected via the tail vein (i.v., bolus). Four 0.4 mg/kg doses of ADC were given every 4 days (Q4Dx4) and PBS was used for the vehicle group (Q4Dx4).

Mice viability and behaviour were recorded every day. Body weights were measured twice a week. The animals were euthanized when a humane endpoint was reached (e.g. 2,000 mm³ tumour volume) or after a maximum of 6 weeks post-dosing.

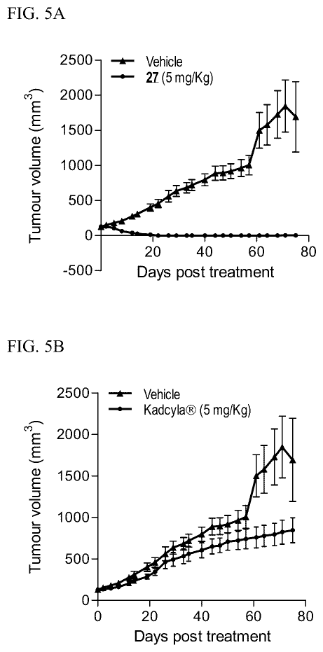

The mean tumour volumes±standard error are represented in FIGS. 4a to 4d for each treatment group. All compounds were well tolerated. Results show that in addition to an initial reduction in tumour volume, conjugate 10 displayed a greater and more prolonged inhibition of tumour growth than conjugate 46 or Adcetris®, whereas the negative control, 32, had no discernible effect over the vehicle (no drug) control.

### Example 15: JIMT-1 Mouse Xenograft Studies Comparing Trastuzumab-Drug Conjugate 27 to Kadcyla® (Comparative)

Trastuzumab-drug conjugate 27 was prepared from reagent 9 by the method described in Example 5.

Healthy female NMRI nude mice (RjOrl:NMRI-Foxn1^{nu}/Foxn1^{nu}) aged 6 weeks at arrival were used for cell inoculation. The animals were maintained in SPF health status according to the FELASA guidelines in housing rooms under controlled environmental conditions.

Tumours were induced by subcutaneous injection of 5×10⁶ JIMT-1 cells (breast carcinoma) in 200 μL of cell suspension in PBS into the right flank. Matrigel (40 μL Matrigel per 200 μL cell suspension) was added shortly before inoculation of tumour cells. Tumours were measured twice a week with calipers, and the volume was estimated using the formula:

$$Tumor Volume ( {mm}^{3} ) = \frac{{width}^{2} \times length}{2}$$

When the tumour volumes reached a mean tumour volume of approximately 150 mm³, the animals were randomised into groups of ten mice (128 mm³ mean tumor volume) and treatment was initiated (Day 0). All test substances were injected via the tail vein (i.v., bolus). A single 5 mg/kg dose of ADC was given in 10 mL/kg and PBS was used for the vehicle group.

Mice viability and behaviour were recorded every day. Body weights were measured twice a week. The animals were euthanized when a humane endpoint was reached (e.g. calculated tumour weight of >10% body weight, animal body weight loss of >20% compared to the body weight at group distribution, ulceration of tumours, lack of mobility, general signs of pain), or at a pre-determined study end date.

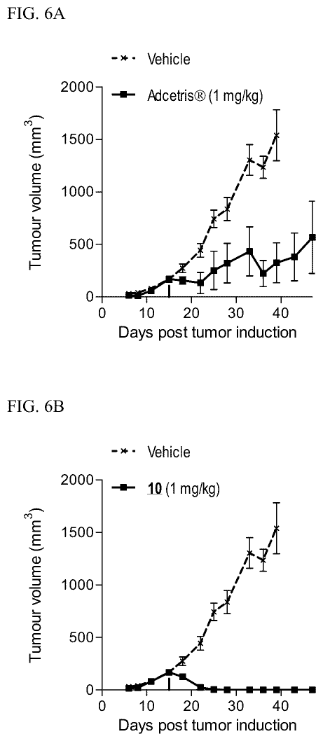

The mean tumour volume±standard error is represented in FIGS. 5a and 5b for each group. Both compounds were well tolerated. Results show that conjugate 27 showed a complete reduction in tumour volume, showing greater activity than the commercial product, Kadcyla®, which had little effect over the vehicle control.

### Example 16: Karpas-299 Mouse Xenograft Study Comparing Brentuximab-Drug Conjugates 10, 28 and 29 to Adcetris® (Comparative)

Conjugates 28 and 29 were prepared from conjugation reagent 21 from Example 12 and conjugation reagent 13 from Example 10, respectively. Conjugations were carried out as described in Example 5.

Healthy female CB17-SCID mice (CBySmn.CB17-Prkdcscid/J, Charles River Laboratories) with an average body weight of 18.9 g were used for cell inoculation (Day 0). 24 to 72 hours prior to tumour cell injection, the mice were γ-irradiated (1.44 Gy, ⁶⁰Co). The animals were maintained in SPF health status according to the FELASA guidelines in housing rooms under controlled environmental conditions.

Tumours were induced by subcutaneous injection of 107 Karpas-299 cells (T-anaplastic large cell lymphoma, ALCL) in 200 μL of RPMI 1640 into the right flank. Tumours were measured twice a week with calipers, and the volume was estimated using the formula:

$$Tumor Volume ( {mm}^{3} ) = \frac{{width}^{2} \times length}{2}$$

Fifteen days after tumour implantation (Day 15), the animals were randomised into groups of eight mice using Vivo Manager® software (169 mm³ mean tumor volume) and treatment was initiated. The animals from the vehicle group received a single intravenous (i.v.) injection of PBS. The treated groups were dosed with a single i.v. injection of ADC at 1 mg/kg.

Treatment tolerability was assessed by bi-weekly body weight measurement and daily observation for clinical signs of treatment-related side effects. Mice were euthanized when a humane endpoint was reached (e.g. 1,600 mm³ tumour volume) or after a maximum of 6 weeks post-dosing.

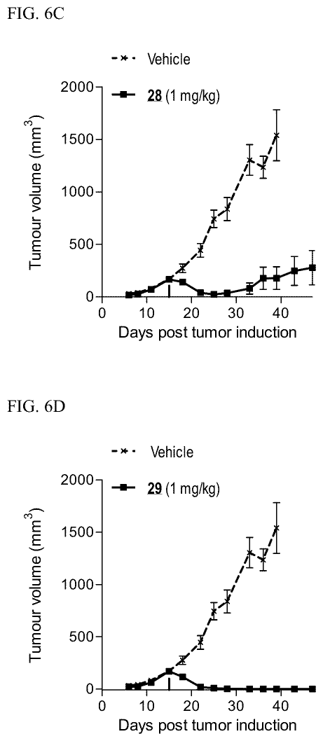

The mean tumour volume±standard error is represented in FIGS. 6a, 6b, 6c and 6d for each group. All compounds were well tolerated. Results show that all conjugates displayed greater activity than Adcetris®, with conjugates 10 and 29 showing a complete reduction in tumour volume for the duration of the study.

### Example 17: Karpas-299 Mouse Xenograft Studies Comparing Brentuximab-Drug Conjugates 10 and 30 to Adcetris® (Comparative)

Conjugate 30 was prepared from conjugation reagent 20 from Example 11. Conjugations were carried out as described in Example 5.

This in vivo study was performed in a similar manner to that described in Example 16.

The mean body weight of the animals at the time of tumour induction (Day 0) was 19.9 g. Randomisation and treatment occurred at Day 15, when the mean tumour volume had reached 205 mm³. The animals from the vehicle group received a single intravenous (i.v.) injection of PBS. The treated groups were dosed with a single i.v. injection of ADC at 1 mg/kg.

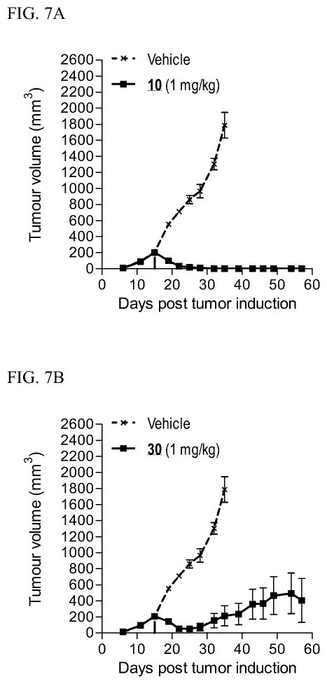

The mean tumour volume±standard error is represented in FIGS. 7a, 7b and 7c for each group. All compounds were well tolerated. Results show that both conjugates 10 and 30 display greater tumour reducing activity than Adcetris®, with 10 displaying complete tumour reduction for the duration of the study.

### Example 18: JIMT-1 Mouse Xenograft Studies Comparing Trastuzumab-Drug Conjugate 31 to Kadcyla® (Comparative)

Conjugate 31 was prepared using conjugation reagent 5 from Example 2. Conjugations were carried out as described in Example 5.

The study was performed in a similar manner to that described in Example 15.

When the tumour volumes reached a mean tumour volume of approximately 150 mm³, the animals were randomised into groups of ten mice (150 mm³ mean tumor volume) and treatment was initiated (Day 0). All test substances were injected via the tail vein (i.v., bolus). A single dose of either 10 mg/kg or 30 mg/kg of ADC was given in 10 mL/kg and PBS was used for the vehicle group.

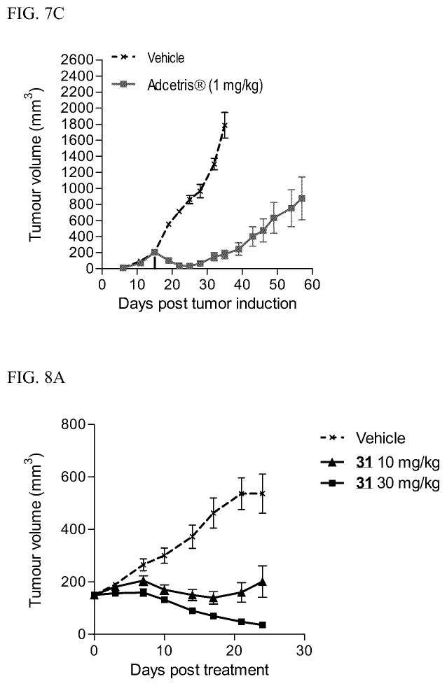

The mean tumour volume±standard error is represented in FIGS. 8a and 8b for each group. All compounds were well tolerated. Results show that conjugate 31 reduces tumour volume, displaying greater activity than Kadcyla® at both doses.

### Example 19: Pharmacokinetic Analysis of ADCs Possessing a Pendant PEG Group, a PEG Group in Series (i.e. Non-Pendant PEG Group) and Adcetris®

Brentuximab conjugate 10 was used in this study as the ADC with a pendant PEG group.

Brentuximab conjugate 11 was used as a comparator with a non-pendant PEG group.

In vivo pharmacokinetics in rats. Sprague-Dawley rats (3 rats per group) with an average weight of 200 g were treated with either 10, 11 or Adcetris®, via the tail vein (IV, bolus) at a single dose of 7 mg/kg. Serial sampling of 100 μL blood was performed before dosing and subsequently at 30 min, 24 h, 48 h, 7 d, 14 d and 35 d post-dosing respectively. Plasma was prepared from fresh blood samples and frozen at −80° C. until analysis.

Total (anti-CD30 antibody) ELISA. Total brentuximab antibody content from plasma samples was quantified using ELISA technology. Briefly, Maxisorp™ 96 well plates (Nunc/Fisher) were coated with recombinant human CD30, (Sino Biological Inc, 2.5 μg/mL in diluent) and incubated at 4° C. overnight. Plasma samples were diluted to ensure that the unknown ADCs fell within the linear range of the sigmoidal standard calibration curve. 100 μL of the diluted plasma sample was then transferred onto the CD30 coated plates and incubated for 3 h. After incubation, plates were washed 3× (200 μL/well) with PBS containing 0.05% Tween-20 (PBST) using a plate washer. HRP conjugated goat anti-human IgG, (Promega) was added to the plate and the samples were incubated for 1 hour at room temperature on a shaker at 350 rpm. The plate was then washed 3× with PBST and once with wash buffer using a plate washer. 100 μL of pre-warmed TMB was added and incubated for 7 minutes. The assay was stopped with 100 μL of 0.5 M H₂SO₄ and read at UV λ=430 nm using the plate reader. Data were plotted and evaluated in GraphPad Prism 5 and Microsoft Excel.

CD30 affinity capture for average DAR determination Affinity capture was performed using streptavidin coated magnetic beads (Dynabeads-Streptavidin TI, Life Technologies). CD30 (Recombinant human CD30, Sino Biological Inc.) was biotinylated and immobilized on beads through streptavidin-biotin binding and finally blocked using skimmed milk peptides. 500 μL of the plasma sample in PBS was added to the CD30-coated beads and incubated overnight at 4° C. and finally washed using PBS. Captured antibodies were eluted using acidic elution buffer for 5 minutes at 4° C. The eluate was subsequently neutralized to pH 7 using sodium acetate buffer, pH 8. Eluted samples were further mixed with HIC loading buffer and analysed using hydrophobic interaction chromatography with UV detection (HIC-UV).

Hydrophobic interaction chromatography for average DAR determination. Affinity captured ADCs were analysed using hydrophobic interaction chromatography in order to determine the average drug to antibody ratio (DAR). The method consisted of a linear gradient from 100% buffer A (50 mM sodium phosphate pH 7.0, 1.5 M ammonium sulfate) to 100% buffer B (50 mM sodium phosphate pH 7.0, 20% isopropanol) in 30 min using a TOSOH TSK gel Butyl-NPR HIC separation column with detection at 280 nm.

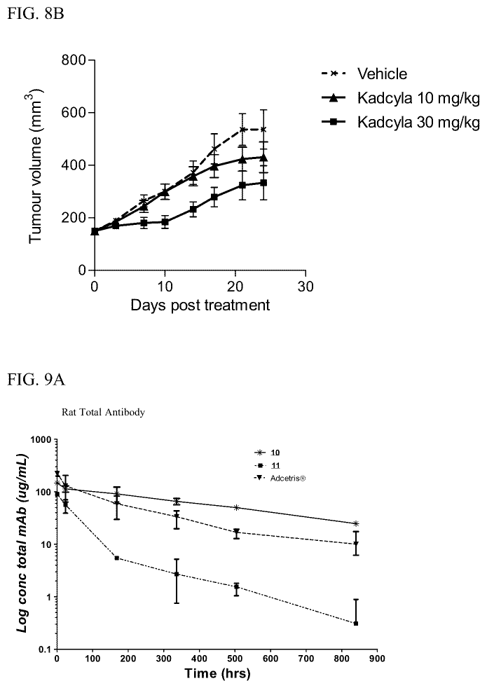

FIGS. 9 a and 9b show total antibody (μg/mL) and average DAR values for ADCs 10, 11 and Adcetris® in rats over a period of 850 h. The results show that conjugate 10 has the lowest rate of drug loss from the conjugate and the lowest rate of clearance from the circulation over this period. Conjugate 11 and Adcetris® (comparatives) show a much faster rate of drug dissociation and are cleared more quickly.

### Example 20: Synthesis of Conjugation Reagent 33 (Comparative) Comprising an Auristatin Cytotoxic Payload

Conjugation reagent 33, which contains a maleimide functional grouping, was synthesised as described within WO2015057699.

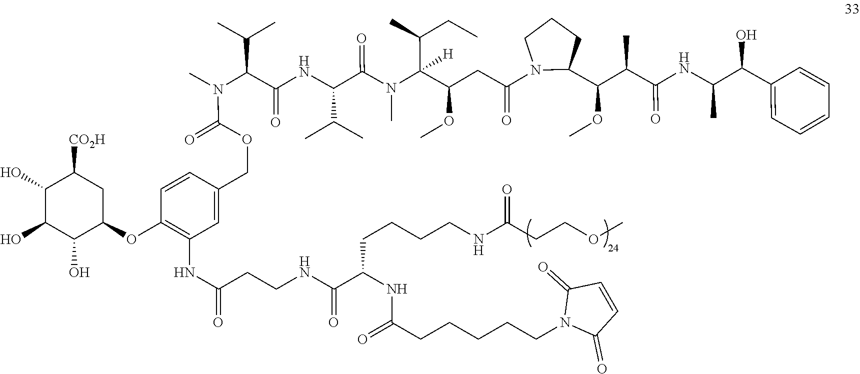

### Example 21: Synthesis of Conjugation Reagent 35 Comprising Cytotoxic Payload 36

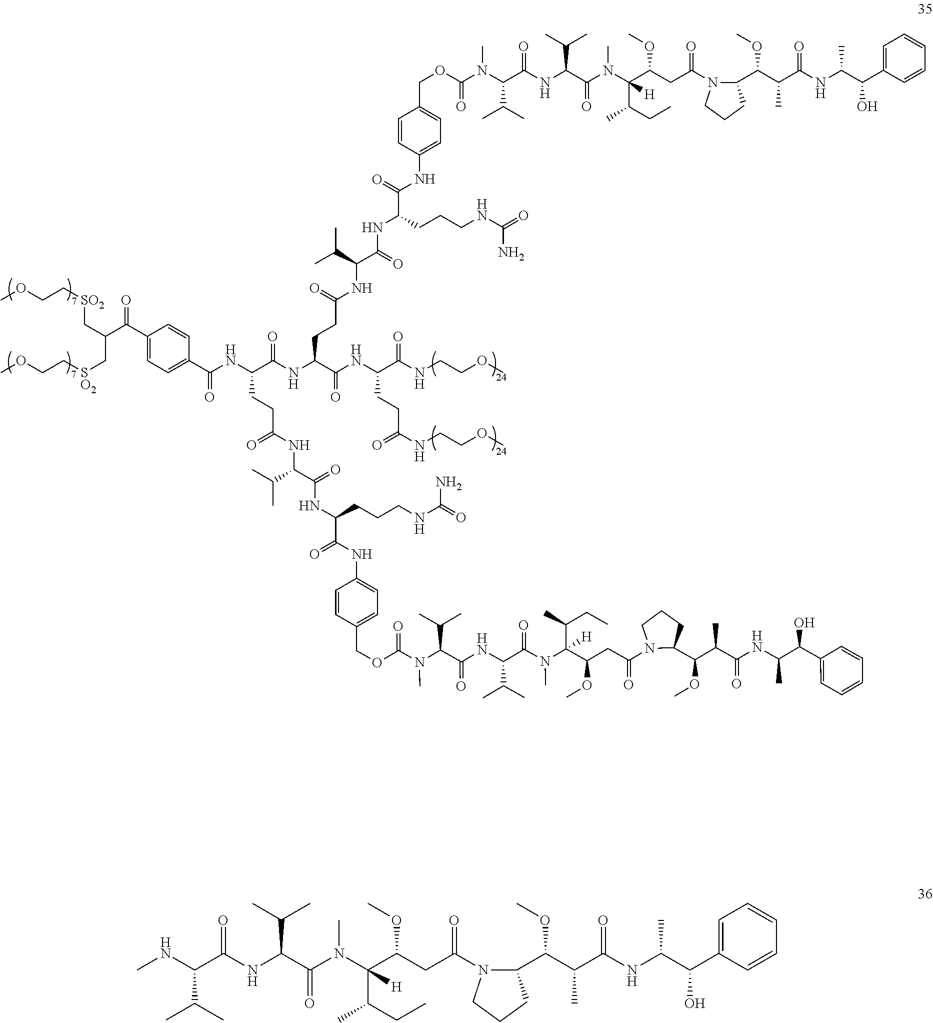

### Step 1: Synthesis of Compound 38.

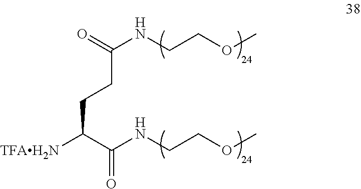

To a stirred solution of Boc-L-Glu(OH)—OH (51.6 mg) in anhydrous DMF (6 mL) was added BOP (277 mg). The solution was stirred at 0° C. for 20 min before MeO-PEG(24)-NH₂ (500 mg) was added followed by NMM (68.9 μL). After 4 h, additional amounts of BOP (92 mg) and NMM (23.0 μL) were added. After a further 2.5 h, the reaction mixture was stored at −20° C. for 18 h before being concentrated in vacuo and purified by reverse phase column C18-column chromatography, eluting with buffer A (v/v): water:0.05% trifluoroacetic acid and buffer B (v/v): acetonitrile:0.05% trifluoroacetic acid (100:0 v/v to 0:100 v/v). The organic solvent was removed in vacuo and the aqueous solvent removed by lyophilisation to give a white solid (373 mg). Formic acid (6 mL) was added to the solid and the resulting mixture stirred under an inert atmosphere for 60 min before being concentrated in vacuo. The residue was dissolved in 95% water:5% acetonitrile:0.05% trifluoroacetic acid (˜6 mL) and lyophilisation overnight to give TFA·H₂N-Glu(PEG(24)-OMe)-PEG(24)-OMe, compound 38, as an off-white solid (330 mg). m/z [M+2H]²⁺ (1144, 5%), [M+3H]³⁺ (763, 35%), [M+4H]⁺ (573, 100%).

### Step 2: Synthesis of Compound 39.

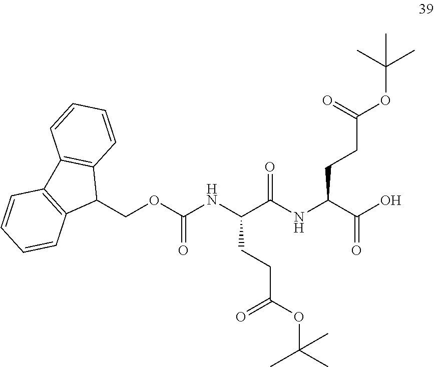 [SMILES: CC(C)(C)OC(=O)CC[C@H](NC(=O)[C@H](CCC(=O)OC(C)(C)C)NC(=O)OCC1c2ccccc2-c2ccccc21)C(=O)O]

To a stirred solution of Fmoc-Glu(OtBu)-OH (2.00 g) in anhydrous DMF (18 mL) at 0° C. was added HOBt (666 mg) and DIC (768 μL). The reaction mixture was allowed to warm to RT and after 2 h, Glu(O^{t}Bu)-OH (1.19 g) and DIPEA (2.46 mL) were added. After stirring for 20 h, the reaction mixture was concentrated in vacuo and purified by reverse phase column C18-column chromatography, eluting with buffer A (v/v): water:0.05% trifluoroacetic acid and buffer B (v/v): acetonitrile:0.05% trifluoroacetic acid (100:0 v/v to 0:100 v/v) The organic solvent was removed in vacuo and the aqueous solvent removed by lyophilisation to give Fmoc-(L-Glu(O^{t}Bu))₂—OH, compound 39, as a white solid (1.03 g). m/z [2M+H]⁺ (1221, 15%), [M+H]⁺ (611, 60%), [M-^{t}Bu+H]⁺ (554, 65%), [M−2^{t}Bu+H]⁺ (499, 100%).

### Step 3: Synthesis of Compound 40.

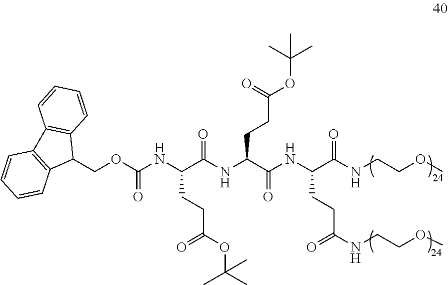

To a stirred solution of compound 38 (330 mg) in anhydrous DMF (10 mL) was added compound 39 (100 mg). At 0° C., HATU (156 mg) and NMM (45.3 L) were then added and the resulting solution stirred for 5 min before further addition of NMM (2.9 μL) and HATU (10.5 mg). The reaction solution was allowed to stir for another 20 min before being warmed to room temperature whereupon stirring was continued for a further 4 h. After this time, additional amounts of HATU (51.1 mg) and NMM (15.1 μL) were added. After a further 1.5 h, the mixture was stored at −20° C. for 18 h before being purified by reverse phase C18-column chromatography, eluting with buffer A (v/v): water:0.05% trifluoroacetic acid and buffer B (v/v): acetonitrile:0.05% trifluoroacetic acid (100:0 v/v to 0:100 v/v). The organic solvent was removed in vacuo and the aqueous solvent removed by lyophilisation to give Fmoc-(L-Glu(O^{t}Bu))₂-L-Glu(PEG(24)-OMe)-PEG(24)-OMe, compound 40, as a white solid (193 mg). m/z [M+3H]³⁺ (961, 20%), [M-^{t}Bu+4H]⁴⁺ (707, 100%), [M−2^{t}Bu+4H]⁴⁺ (693, 85%), [M+5H]⁵⁺ (577, 75%).

### Step 4: Synthesis of Compound 41.

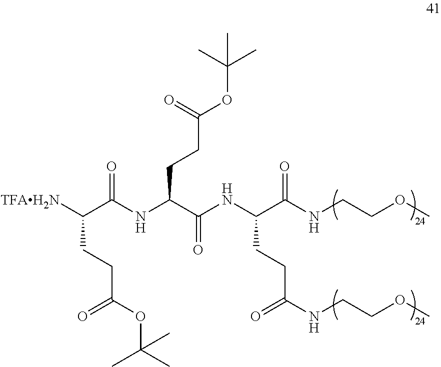

To a stirred solution of 41 (193 mg) in anhydrous DMF (1.5 mL) was added piperidine (19.8 μL). After 90 min, a further amount of piperidine (13.2 μL) was added and the reaction stirred for another 90 min before being stored at −20° C. for 18 h. The solvent was removed under high vacuum and the resulting residue triturated in hexane. The residue was further dried under high vacuum for 30 min before being dissolved in a 50:50 mixture of buffer A:buffer B (2 mL, buffer A (v/v): water:0.05% trifluoroacetic acid and buffer B (v/v): acetonitrile:0.05% trifluoroacetic acid) and lyophilised overnight to give TFA·H₂N-(L-Glu(O^{t}Bu))₂-L-Glu(PEG(24)-OMe)-PEG(24)-OMe, compound 41, as a pale blue solid (186 mg). m/z [M+3H]³⁺ (887, 20%), [M+4H]⁴⁺ (666, 100%), [M+5H]⁵⁺ (533, 30%).

### Step 5: Synthesis of Compound 42.

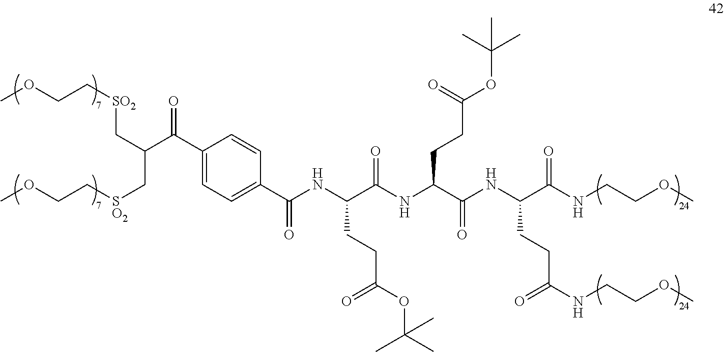

To a stirred solution of 4-[2,2-bis[alpha-methoxy-omega-sulfonyl hepta(ethylene glycol)]acetyl]benzoic acid (71.0 mg) in anhydrous DMF (1.5 mL) was added to HATU (27.9 mg). The mixture was cooled to 0° C. and stirred under an inert atmosphere for 30 min. A solution of 41 (186 mg) in anhydrous DMF (2.5 mL) was added, followed by HATU (22.9 mg) and NMM (14.7 μL), and the mixture allowed to warm to RT.

After 3 h, additional amounts of 4-[2,2-bis[alpha-methoxy-omega-sulfonyl hepta(ethylene glycol)]acetyl]benzoic acid (18.1 mg), HATU (50.8 mg) and NMM (15.1 μL) were added. After a further 1.5 h, further amounts of 4-[2,2-bis[alpha-methoxy-omega-sulfonyl hepta(ethylene glycol)]acetyl]benzoic acid (9.04 mg), HATU (50.8 mg) and NMM (15.1 μL) were added. The reaction mixture was stirred for a further 8 h and purified twice by reverse phase C18-column chromatography, eluting with buffer A (v/v): water:0.05% trifluoroacetic acid and buffer B (v/v): acetonitrile:0.05% trifluoroacetic acid (100:0 v/v to 0:100 v/v). The organic solvent was removed in vacuo and the aqueous solvent removed by lyophilisation to give 42, as a pale yellow oil (assumed quantitative). m/z [M+4H]⁴⁺ (902, 60%), [M-^{t}Bu+4H]⁴⁺ (888, 60%), [M−2^{t}Bu+4H]⁴⁺ (874, 45%), [M-^{t}Bu+5H]⁵⁺ (711, 100%).

### Step 6: Synthesis of Compound 43.

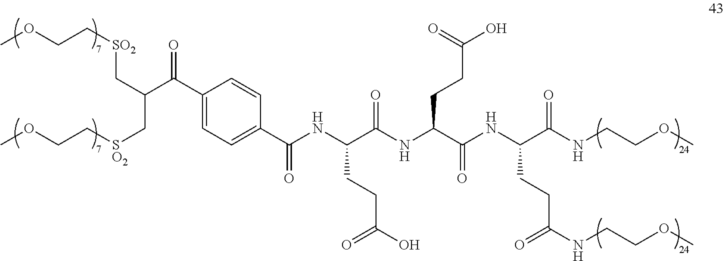

Formic acid (2 mL) was added to 42 under an inert atmosphere. The reaction mixture was stirred for 60 min before being concentrated in vacuo. The material was purified by reverse phase C18-column chromatography, eluting with buffer A (v/v): water:0.05% trifluoroacetic acid and buffer B (v/v): acetonitrile:0.05% trifluoroacetic acid (100:0 v/v to 0:100 v/v).

The organic solvent was removed in vacuo and the aqueous solvent removed by lyophilisation to give 43, as a colourless oil (28.1 mg). m/z [M+3H]³⁺ (1165, 5%), [M+4H]⁴⁺ (874, 65%), [M+5H]⁵⁺ (699, 100%).

### Step 7: Synthesis of Reagent 35.

To a stirred solution of 43 (15.0 mg) in anhydrous DMF (270 μL) was added HATU (4.08 mg). The mixture was cooled to 0° C. and stirred under an inert atmosphere for 20 min. A solution of val-Cit-PAB-MMAE (12.2 mg) in anhydrous DMF (300 μL) was added, followed by HATU (2.45 mg) and NMM (1.89 μL), and the mixture allowed to warm to RT. After 4 h 20 min, additional amounts of HATU (3.3 mg) and NMM (0.94 μL) were added. The reaction mixture was stirred for a further 2 h before being stored at −20° C. for 18 h. Upon warming to RT, HATU (3.26 mg) and NMM (0.94 μL) were added to the stirred solution. After a 4.5 h, additional amounts of HATU (1.63 mg) and NMM (0.472 μL) were added and the reaction allowed to stir for a further 2.5 h before being stored at −20° C. for 18 h. The material was purified by reverse phase C18-column chromatography, eluting with buffer A (v/v): water:0.05% trifluoroacetic acid and buffer B (v/v): acetonitrile:0.05% trifluoroacetic acid (100:0 v/v to 0:100 v/v). The organic solvent was removed in vacuo and the aqueous solvent removed by lyophilisation to give 35, as a white solid (13.8 mg). m/z [M+4H]⁴⁺+(1426, 5%), [M+5H]⁵⁺ (1141, 70%), [M+6H]⁶⁺ (951, 100%), [M+7H]⁷⁺ (815, 20%).

### Example 22: Conjugation of Reagents 33 (Comparative) and 35 to Brentuximab to Give Antibody-Drug Conjugates (ADCs) 34 (Comparative) and 44

Conjugation reagent 33 was conjugated to Brentuximab, giving rise to ADC 34 using the methods described within WO2015057699, U.S. Pat. No. 7,090,843, Lyon et al., (2015) Nature Biotechnology, 33(7) p 733-736 and Lyon et al., (2012), Methods in Enzymology, Volume 502, p 123-137. Briefly, Brentuximab in 20 mM sodium phosphate buffer, pH 6.5 (150 mM NaCl, 20 mM EDTA) was reduced with TCEP (6 eq.) at 40° C. for 1 h. Conjugation of the reduced antibody with 2.0 molar equivalents of reagent 33 per free thiol was then performed). Reagent 33 was added to the mAb to give a final antibody concentration of 4 mg/mL. The solution was mixed gently and incubated at 22° ° C. for 2 h. After 2 h additional reagent 33 (0.2 molar equivalents) was added and the mixture was incubated for a further 1 h at 22° C. Excess reagent 33 was quenched with N-acetyl-L-cysteine (20 eq. over reagent) and the crude reaction was purified using a 1 mL ToyoPearl Phenyl-650S HIC column equilibrated with 50 mM sodium phosphate, pH 7 (2 M NaCl). The ADC was eluted from the column with a gradient of 50 mM sodium phosphate, pH 7 (20% isopropanol). Fractions containing ADC were pooled and concentrated (Vivaspin20, 10 kDa PES membrane) to give an average DAR 8 product. The concentrated sample was buffer exchanged into DPBS, pH 7.1-7.5 and sterile filtered.

ADC 34 was difficult to purify and characterise due to the heterogeneity of the reaction products (number of DAR variants), leading to poor resolution of the individual DAR species by preparative HIC. Results showed that the final reaction product contained significant quantities of DAR species both greater than and less than DAR 8. Purifying the DAR8 species completely from these higher and lower than DAR8 species would result in significantly lower yields of DAR8 in the final product.

Conjugation reagent 35 was conjugated to Brentuximab, giving rise to ADC 44 using the conjugation procedure described in Example 5.

### Example 23: Karpas-299 Mouse Xenograft Study Comparing Brentuximab-Drug Conjugates 34 and 44

This in vivo study was performed in a similar manner to that described in Example 16. The mean body weight of the animals at the time of tumour induction (Day 0) was 18.8 g. The number of animals per treatment group was 5. Randomisation and treatment occurred at Day 17, when the mean tumour volume had reached 167 mm³. The animals from the vehicle group received a single intravenous (i.v.) injection of PBS. The treated groups were dosed with a single i.v. injection of ADC at 0.5 mg/kg. All compounds were well tolerated.

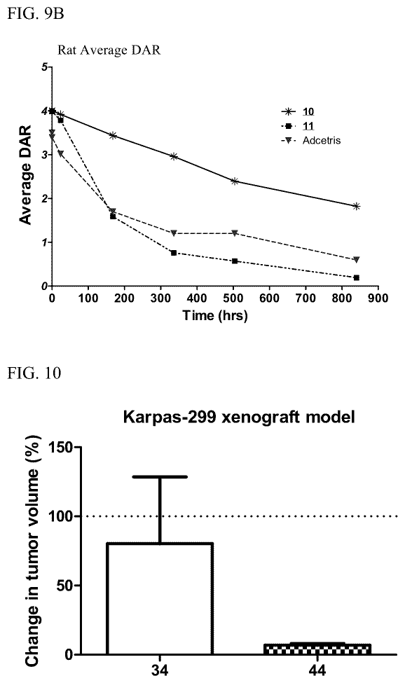

FIG. 10 shows the % change in tumour volume for both ADCs 34 and 44 at Day 28. 100% was defined as the tumour volume at the first day of dosing (Day 17).

### Example 24: Comparison of Pendant PEG Conjugates 34 (Comparative) and 44 by Thermal Stress Test

ADC samples 34 and 44 were each prepared at 0.5 mg/mL by dilution with DPBS pH 7.1-7.5.

The two ADC samples were incubated at 65° C. for 30 minutes followed by incubation in an ice bath for 5 min before Size Exclusion Chromatography (SEC). SEC was performed using a TOSOH Bioscience TSK gel Super SW 3000 column. UV absorbance at 280 nm was monitored during an isocratic elution with a 2 M Potassium phosphate buffer, pH 6.8 (0.2 M potassium chloride and 15% isopropanol).

Tables 4a and 4b below show the conformations of ADCs 34 and 44 before and after thermal stress test, as measured by the area under the curve of each peak by Abs 280 nm, following SEC.

The results in Tables 4a and 4b show that ADC 44 remains in a non-aggregated state to a much greater extent than ADC 34 following thermal stress test. In addition, the results also show that 34 dissociates into lighter molecular weight components to a greater extent than conjugate 44.

TABLE 4a

ADC conformation before thermal
stress test (% of total ADC)

| | 34 | 44 |
|---|---|---|
| Non-aggregated | 97.4 | 100 |
| Aggregated | 1.8 | 0 |
| Dissociated | 0.7 | 0 |

TABLE 4b

ADC conformation after thermal
stress test (% of total ADC)

| | 34 | 44 |
|---|---|---|
| Non-aggregated | 11.1 | 69.4 |
| Aggregated | 71 | 27.7 |
| Dissociated | 17.9 | 1.2 |

### Example 25: Comparison of Average DARs for Pendant PEG Conjugates 34 and 44 Following Incubation within Human Serum

ADCs 34 and 44 were diluted to 0.1 mg/mL in human serum, 88% (v/v) serum content. Each solution was immediately sub-aliquoted into 4×0.5 mL low-bind Eppendorf tubes. Two of the Eppendorf tubes, corresponding to the ‘0’ time points were immediately transferred to the −80° C. freezer, whereas the rest of the samples were incubated at 37° C. for 6 days. After 6 days the appropriate samples were removed from the freezer and incubator for purification by affinity capture (CD30-coated magnetic beads), followed by analysis using hydrophobic interaction chromatography (HIC). CD30 affinity capture and HIC for average DAR determination were carried out as described in Example 19.

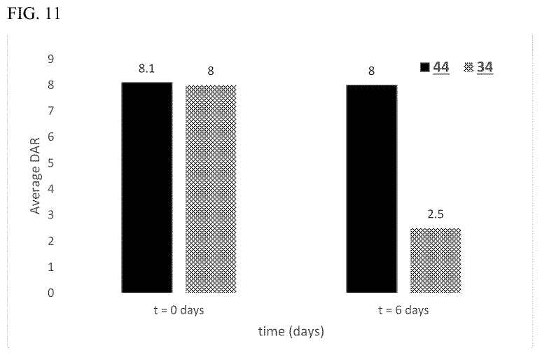

FIG. 11 shows that after 6 days at 37° ° C. in human serum, conjugate 34 has lost much of its cytotoxic payload, whereas conjugate 44 remains largely unchanged, as indicated by the reduction in average DAR value of the sample. This indicates that the conjugate of the invention would have improved stability in vivo.

### Example 26: Synthesis of Conjugation Reagent 45 (Comparative) Comprising an Auristatin Cytotoxic Payload

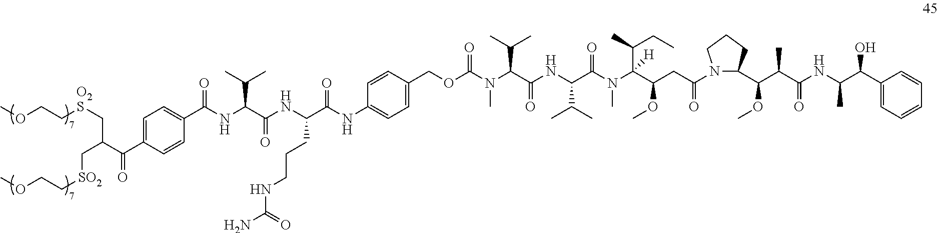

To the TFA salt of val-cit-PAB-MMAE salt having the structure below:

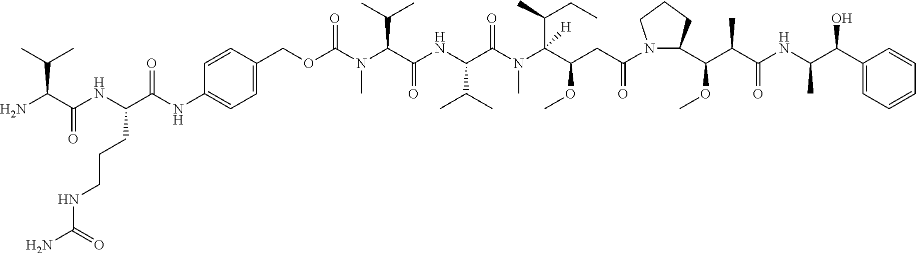 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C]

(25.0 mg) was added a solution of reagent 5A (15.6 mg) in DMF (1.5 mL) and stirred under an inert nitrogen atmosphere at room temperature for 5 min. The mixture was cooled to 0° C. and aliquots of HATU (6.1 mg) and NMM (1.8 μL) were added every 20 min for a total of 5 additions. After 1.5 h, the reaction mixture was warmed to room temperature. After 2 h, volatiles were removed in vacuo. The resulting residue was dissolved in water and acetonitrile (v/v; 1/1, 0.6 mL), and purified by reverse phase C18-column chromatography eluting with buffer A (v/v): water:5% acetonitrile:0.05% trifluoroacetic acid and buffer B (v/v): acetonitrile:0.05% trifluoroacetic acid (100:0 v/v to 0:100 v/v). The organic solvent was removed in vacuo and the aqueous solvent was removed by lyophilisation to give bis-mPEG(7u)sulfone-propanoyl-benzamide-val-cit-PAB-MMAE reagent 45 as a white powder (22.4 mg, 68%) m/z [M+2H²⁺] 1035.

1-24. (canceled)

25. A conjugate of an antibody or antibody fragment with a therapeutic, diagnostic or labelling agent, said conjugate being a conjugate formed from the conjugation of an antibody or antibody fragment with a conjugating reagent selected from conjugation reagent 1: 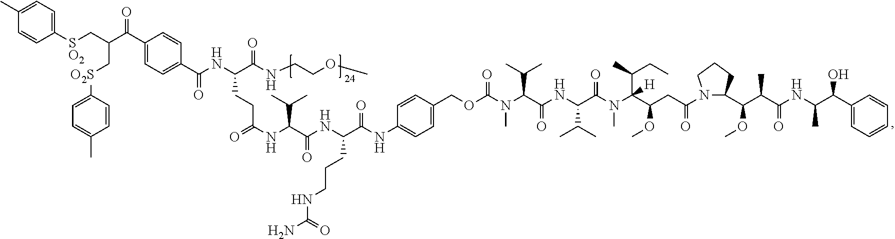
conjugation reagent 5: 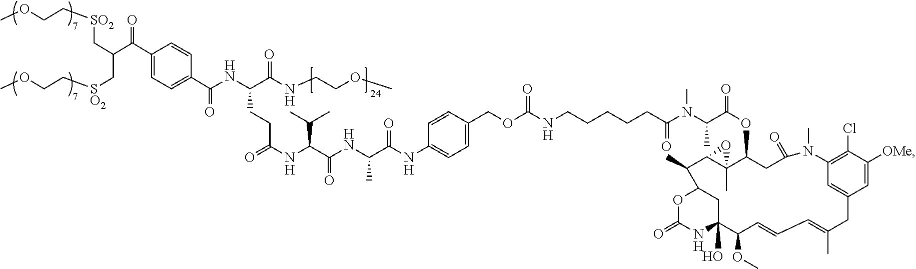
conjugation reagent 8: 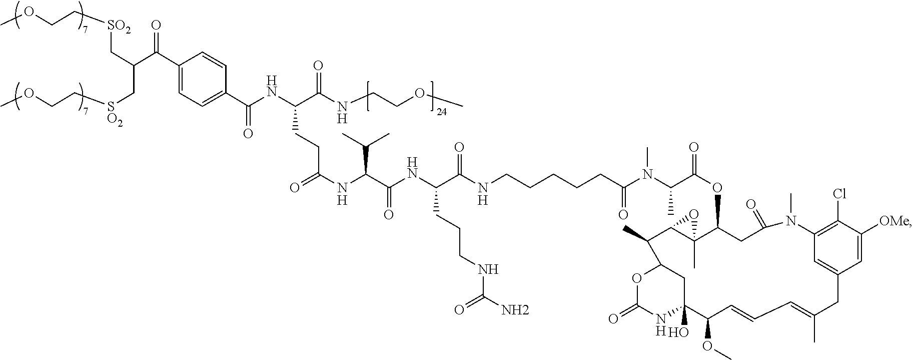
conjugation reagent 9: 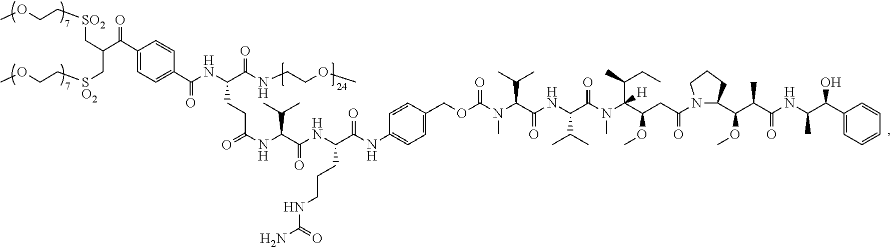
conjugation reagent 11A: 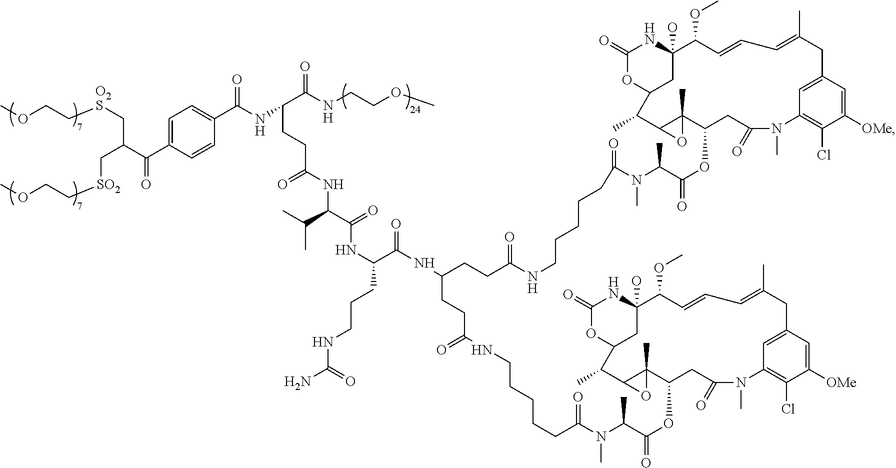
conjugation reagent 13: 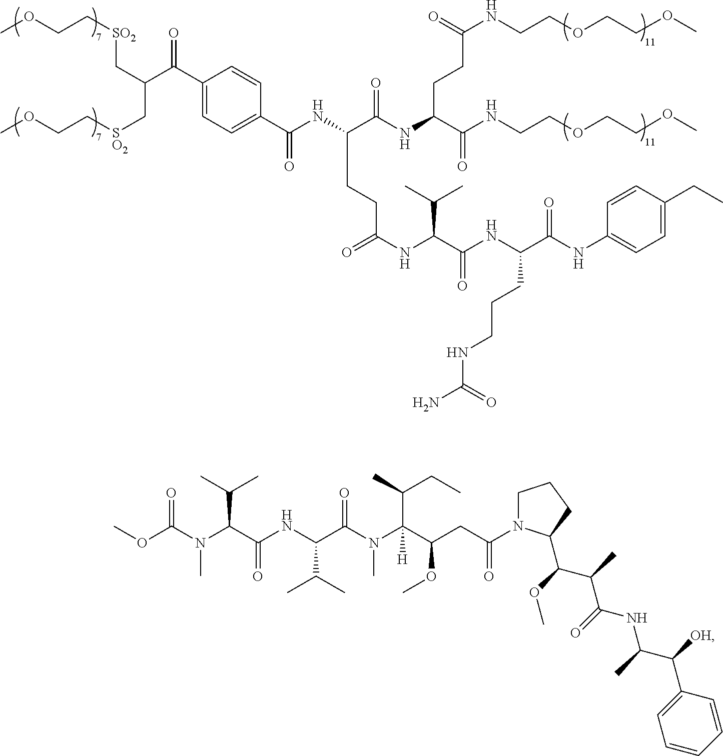
conjugation reagent 16A: 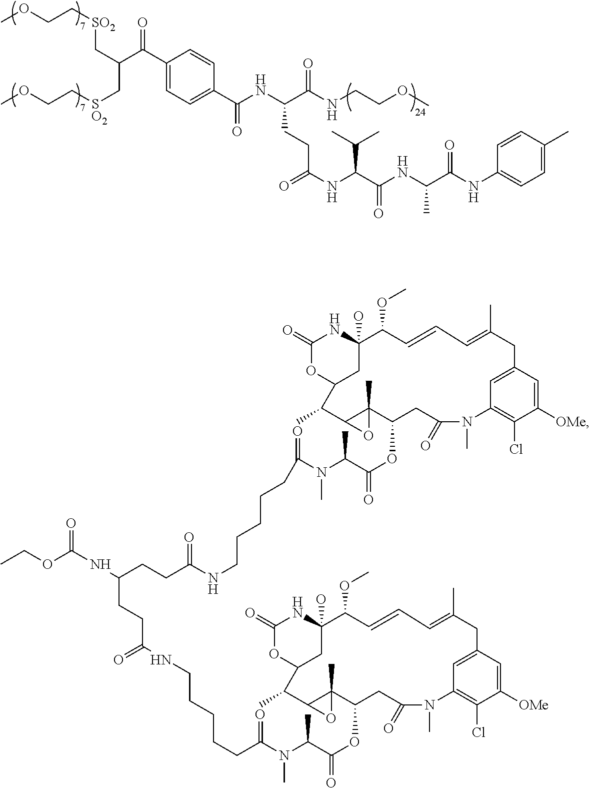
conjugation reagent 20: 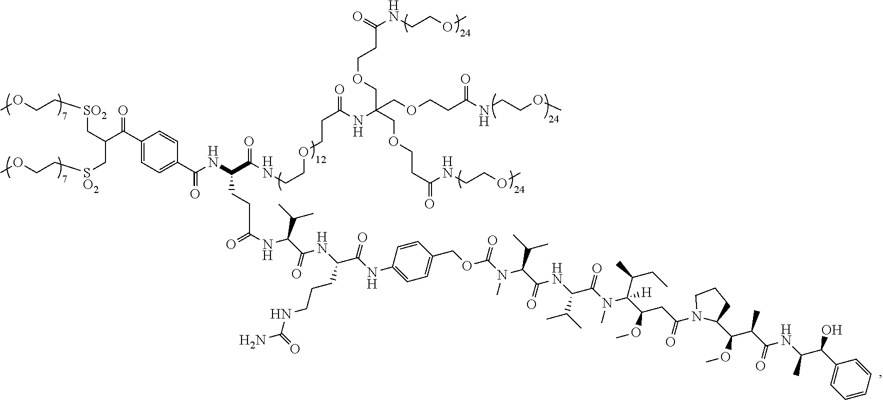
conjugation reagent 21: 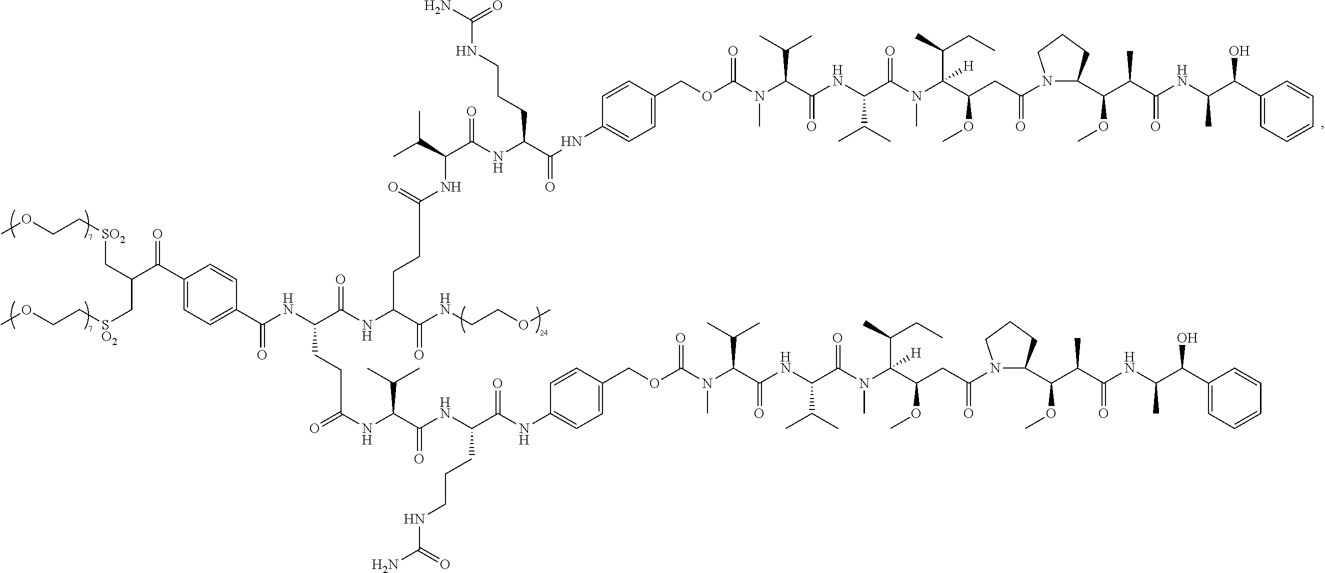
and
conjugation reagent 35:

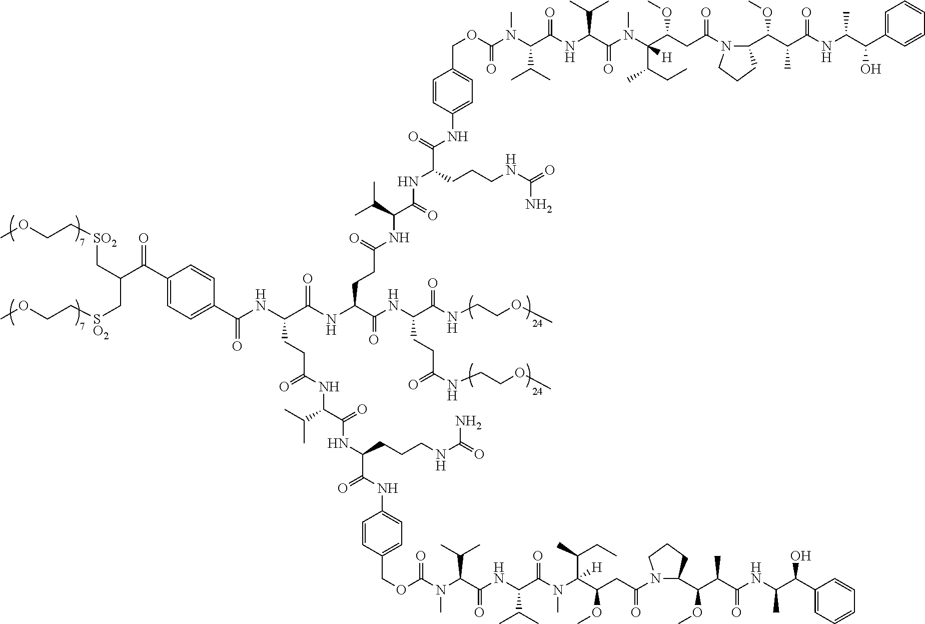

26. The conjugate of claim 25, wherein the conjugation reagent is conjugation reagent 9.

27. The conjugate of claim 26, wherein the antibody or antibody fragment is brentuximab.
